(19) United States
(12) Patent Application Publication
(10) Pub. No.: US 2023/0285542 A1
(43) Pub. Date: Sep. 14, 2023

(54) Coronavirus Vaccine

(71) Applicant: Texas Tech University System, Lubbock, TX (US)

(72) Inventors: Harvinder Singh Gill, Lubbock, TX (US); Gaurav Joshi, Lubbock, TX (US)

(21) Appl. No.: 18/006,097

(22) PCT Filed: Jul. 22, 2021
(86) PCT No.: PCT/US2021/042692

§ 371 Date: Jan. 19, 2023

Related U.S. Application Data
(60) Provisional application No. 63/054,938, filed on Jul. 22, 2020.

(51) Int. Cl. A61K 39/215; A61K 39/39; A61K 39/155; A61K 39/145; A61K 39/235; A61K 39/125; A61K 39/12; A61P 31/14

(52) U.S. Cl. CPC A61K 39/215; A61K 39/39; A61K 39/155; A61K 39/145; A61K 39/235; A61K 39/125; A61K 39/12; A61P 31/14; A61K 2039/55572

(57) ABSTRACT

The present invention includes and immunogenic composition and methods of immunizing a mammal or avian comprising: a nanoparticle conjugated to one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response. In certain embodiments the antigenic peptides or fusion polypeptides are selected from at least one of SEQ IN NOS: 1 to 16, 22 to 39, or any combination thereof.

### CROSS-REFERENCE TO RELATED APPLICATIONS

This application claims priority to U.S. Provisional Application Ser. No. 63/054,938, filed Jul. 22, 2020, the entire contents of which are incorporated herein by reference.

### STATEMENT OF FEDERALLY FUNDED RESEARCH

This invention was made with government support under R01AI137846 awarded by National Institute of Allergy and Infectious Diseases of the National Institutes of Health. The government has certain rights in the invention.

### TECHNICAL FIELD OF THE INVENTION

The present invention relates in general to the field of immunization against viruses, and in particular, to a novel coronavirus vaccine.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on ______, 2021, is named ______.txt and is ______ bytes in size.

### BACKGROUND OF THE INVENTION

Without limiting the scope of the invention, its background is described in connection with virus vaccines.

Coronaviruses are inherently diverse. Coronaviruses (CoVs) belong to the family Coronaviridae within the Nidovirales order. As the name ‘corona’ indicates, the CoVs have a characteristic crown-like appearance on their outer surface due to the spike protein, which facilitates the entry of the virus into host cells. The CoV family is comprised of four genera: alpha, beta, gamma, and delta CoVs[1]. Alpha- and beta-CoVs are able to infect diverse species such as mammals, cats, bats, mice, pigs, and humans[2-8], while gamma and delta-CoVs generally infect birds, but few of them could infect mammals as well[9-12]. Such a broad diversity of infectivity and reservoir species greatly increase the chance of spillover of the CoV from animals and birds to humans, which has proven to be the case for the three CoV outbreak within the last 18 years.

Different coronaviruses have infected humans so far. So far, seven CoVs, including the 2019 pandemic strain have been known to infect humans. The human coronaviruses (HCoVs) belong to two of these genera, namely the alpha coronaviruses (includes HCoV-229E and HCoV-NL63)[13, 14] and beta coronaviruses (includes HCoV-HKU1, HCoV-OC43, the severe acute respiratory syndrome coronavirus: SARS-CoV, Middle East respiratory syndrome coronavirus: MERS-CoV and novel corona virus 2019: 2019-nCoV or SAR-CoV-2) [15-22]. Amongst these, three strains have caused severe infections in humans; SARS-CoV (˜10% mortality rate) originated from China in 2002[15], MERS-CoV (˜34.4% mortality rate) originated from Saudi Arabia in 2012[18], and the newly identified 2019-nCoV SAR-CoV2 (˜4.4% mortality rate [23] originated from Wuhan, China in December 2019[21, 24], which has now become a pandemic. Until Jul. 14, 2020, pandemic 2019-nCoV has infected more than 13 million people worldwide and has led to 576,752 deaths and continues to pose significant threat to global public health. The origin of SARS-CoV and MERS-CoV is thought to be bats. Genome sequences of 2019-nCoV match 79.5% and 96% similarity at nucleotide level to the SARS-CoV and bat CoVs, respectively, which suggests that the nCoV also originated in bats.

What is needed is a novel, highly effective vaccine that is easy to produce and that will have minimal or no immunity to non-coronavirus portions of the vaccine. Also, needed is a vaccine that will provide long-term immunity, that triggers the production of blocking antibodies, and that these blocking antibodies are found in various secretions, such as alveolar mucus, saliva, nasal mucus, sweat, feces, etc., and these antibodies should be able to neutralize, e.g., at least one type of CoV, and preferably more than one type of CoV.

### SUMMARY OF THE INVENTION

In one embodiment, the present invention includes an immunogenic composition comprising: a nanoparticle conjugated to one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response. In one aspect, the formulation further comprises an adjuvant selected from at least one of: monophosphoryl lipid A, synthetic lipid A, lipid A mimetics or analogs, aluminum salts, cytokines, saponins, muramyl dipeptide, N-glycolyl dipeptide, polyIC, polyCpG, lipopolysaccharide, polyphosphazenes, emulsions, virosomes, virus like particles, bacteria, algae, yeast, cochleates, poly(lactide-co-glycolides) microparticles, poloxamer particles, microparticles, toll-like receptor agonists, helper T cell agonists, T cell stimulating peptides added to the composition, T cell stimulating peptides conjugated or fused to the one or more antigenic peptides or fusion polypeptides, water in oil emulsion, oil in water emulsion, resiquimod, inulin, algammulin, lipid particles, or liposomes. In another aspect, the formulation further comprises one or more spherical particles or any other regular or irregular shape with a mean of its largest dimension being below 1000 micrometers, below 100 micrometers, and more preferably below 10 micrometers, and below 1 micrometer, and below 0.5 micrometer, and the deviation of the particles being less than 75% of the mean, less than 50% of the mean, less than 25% of the mean or less than 15% of the mean. In another aspect, the nanoparticle comprises the antigenic peptides or polypeptides, wherein the antigenic peptides or polypeptides are crosslinked, precipitating the antigenic peptides or polypeptides, aggregating the antigenic peptides or polypeptides, the particle is made of a different material such as metals or their oxides or their salts (gold, silver, iron oxide, aluminum hydroxide, aluminum phosphate), synthetic polymers (poly(lactide-co-glycolide), polycapralactone, polyanhydrides), inorganic molecules (silica), metal particles, zoonotic viruses, human viruses, bacterial viruses, plant viruses, bacteria, bacterial or fungal spores, yeast, liposomes, lipids, or other proteins and peptides that self-assemble, DNA/RNA molecules that self-assemble, pollen shells, carbohydrates, sugars, virus-like particles, or any combination of the aforementioned, a mixture of heterogenous particles made from one or more materials, the materials combined with a coating or a polymer layer on the metal particles, or by coating gold over silica particles, or combinations thereof. In another aspect, the antigenic peptides or polypeptides are mixed with particles, the antigenic peptides or polypeptides are chemically attached to a particle surface, or the antigenic peptides or polypeptides are entrapped in the particle core, or a combination thereof, wherein the antigenic peptides or polypeptides are in a free form and in an attached form. In another aspect, the one or more antigenic peptides or fusion polypeptides are made synthetically, recombinantly, in a prokaryotic expression system or a eukaryotic expression system.

In another aspect, the composition is formulated for a mucosal, intranasal, intramuscular, intravenous, intrapulmonary, enteric, oral, subcutaneous, intradermal, subdermal, or transdermal route of administration. In another aspect, the one or more antigenic peptides or fusion polypeptides are expressed in a prokaryotic expression system or a eukaryotic expression system. In another aspect, the one or more antigenic peptides or fusion polypeptides are separated by a linker. In another aspect, the one or more antigenic peptides or fusion polypeptides are selected from at least one of SEQ ID NOS: 1 to 16, 22 to 39, any combination and concatemers thereof. In another aspect, the composition elicits two or more immune responses selected from: immunoglobulin production, Th1 protective immunity, Th2 protective immunity, or any combination thereof. In another aspect, the composition further comprises a buffer selected from the group consisting of phosphate buffer, citrate buffer, phosphate citrate buffer, borate buffer, tris(hydroxymethyl)aminomethane (Tris) containing buffer, succinate buffer, and buffers containing glycine or histidine as one of the buffering agents. In another aspect, the composition is in a liquid or a lyophilized form. In another aspect, the composition is contained within pre-filled syringes, microneedle patch, needle-free patch, and/or inhalation or nasal sprays. In another aspect, the virus is selected from a rhinovirus, coronavirus, paramyxoviridae, Orthomyxoviridae, adenovirus, parainfluenza virus, metapneumovirus, respiratory syncytial virus or influenza virus.

In another embodiment, the present invention includes a method of eliciting protective immunity to a viral infection in a mammal or avian comprising administering to the mammal or avian a vaccine comprising a nanoparticle conjugated to one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response. In one aspect, the formulation further comprises at least one of: monophosphoryl lipid A, synthetic lipid A, lipid A mimetics or analogs, aluminum salts, cytokines, saponins, muramyl dipeptide, N-glycolyl dipeptide, polyIC, polyCpG, lipopolysaccharide, polyphosphazenes, emulsions, virosomes, virus like particles, bacteria, algae, yeast, cochleates, poly(lactide-co-glycolides) microparticles, poloxamer particles, microparticles, toll-like receptor agonists, helper T cell agonists, T cell stimulating peptides added to the composition, T cell stimulating peptides conjugated or fused to the one or more antigenic peptides or fusion polypeptides, water in oil emulsion, oil in water emulsion, resiquimod, inulin, algammulin, lipid particles, or liposomes. In another aspect, the formulation further comprises one or more spherical particles or any other regular or irregular shape with a mean of its largest dimension being below 1000 micrometers, below 100 micrometers, and more preferably below 10 micrometers, and below 1 micrometer, and below 0.5 micrometer, and the deviation of the particles being less than 75% of the mean, less than 50% of the mean, less than 25% of the mean or less than 15% of the mean. In another aspect, the nanoparticle comprises the antigenic peptides or polypeptides, wherein the antigenic peptides or polypeptides are crosslinked, precipitating the antigenic peptides or polypeptides, aggregating the antigenic peptides or polypeptides, the particle is made of a different material such as metals or their oxides or their salts (gold, silver, iron oxide, aluminum hydroxide, aluminum phosphate), synthetic polymers (poly(lactide-co-glycolide), polycapralactone, polyanhydrides), inorganic molecules (silica), metal particles, zoonotic viruses, human viruses, bacterial viruses, plant viruses, bacteria, bacterial or fungal spores, yeast, liposomes, lipids, or other proteins and peptides that self-assemble, DNA/RNA molecules that self-assemble, pollen shells, carbohydrates, sugars, virus-like particles, or any combination of the aforementioned, a mixture of heterogenous particles made from one or more materials, the materials combined with a coating or a polymer layer on the metal particles, or by coating gold over silica particles, or combinations thereof. In another aspect, the antigenic peptides or polypeptides are mixed with particles, the antigenic peptides or polypeptides are chemically attached to a particle surface, or the antigenic peptides or polypeptides are entrapped in the particle core, or a combination thereof, wherein the antigenic peptides or polypeptides are in a free form and in an attached form. In another aspect, the one or more antigenic peptides or fusion polypeptides are made synthetically, recombinantly, in a prokaryotic expression system or a eukaryotic expression system. In another aspect, the one or more antigenic peptides or fusion polypeptides are separated by a linker. In another aspect, the one or more antigenic peptides or fusion polypeptides are selected from at least one of SEQ ID NOS: 1 to 16, 22 to 39, any combination and concatemers thereof. In another aspect, the composition is formulated for a mucosal, intranasal, intramuscular, intravenous, intrapulmonary, enteric, oral, subcutaneous, intradermal, subdermal, or transdermal route of administration. In another aspect, the one or more antigenic peptides or polypeptides are expressed in a prokaryotic expression system or a eukaryotic expression system. In another aspect, the composition elicits two or more immune specific responses selected from: immunoglobulin production, Th1 protective immunity, Th2 protective immunity, or any combination thereof. In another aspect, the method further comprises adding a buffer selected from the group consisting of phosphate buffer, citrate buffer, phosphate citrate buffer, borate buffer, tris(hydroxymethyl)aminomethane (Tris) containing buffer, succinate buffer, and buffers containing glycine or histidine as one of the buffering agents. In another aspect, the composition is in a liquid or a lyophilized form. In another aspect, the composition is contained within pre-filled syringes, microneedle patch, needle-free patch, and/or inhalation or nasal sprays. In another aspect, the vaccine is in a dose amount of from about 1 microgram to about 1 gram. In another aspect, the virus is selected from a rhinovirus, coronavirus, paramyxoviridae, Orthomyxoviridae, adenovirus, parainfluenza virus, metapneumovirus, respiratory syncytial virus or influenza virus. In another aspect, the one or more antigenic peptides or polypeptides comprise 2, 3, 4, 5, 6, 7, 8, 9, 10, 11, 12, 13, 14, 15, 16 or more antigenic peptides or polypeptides.

In another embodiment, the present invention includes an immunogenic formulation comprising: a peptide or fusion polypeptide comprising: (Aₙ₁Bₙ₂Cₙ₃Dₙ₄Eₙ₅Fₙ₆Gₙ₇Hₙ₈Iₙ₉Jₙ₁₀Kₙ₁₁)ₙ₁₂ wherein:

| Name | Protein | SEQ ID NO: |
|---|---|---|
| A | Spike_1 | 1 |
| B | Spike_2 | 2 |
| C | Spike_3 | 3 |
| D | Spike_4 | 4 |
| E | Spike_5 | 5 |
| F | Matrix_1 | 6 |
| G | NP_1 | 7 |
| H | RDRP_1 | 8 |
| I | RDRP_2 | 9 |
| J | RDRP_3 | 10 |
| K | RDRP_4 | 11 |

wherein n1, n2, n3, n4, n5, n6, n7, n8, n9, n10, and n11 can be any digit greater than or equal to zero but all are not simultaneously equal to zero, and the order of A, B, C, D, E, F, G, H, I, J, and K can be in any permutation and combination and wherein n12 is greater than zero, wherein the peptides are optionally separated by a linker. In one aspect, the formulation further comprises one or more atoms or one or more molecules are placed at the amino terminus, the carboxy terminus, between one or more amino acids, between one or more one or more peptides, or any combination thereon. In another aspect, the formulation further comprises one or more spherical particles or any other regular or irregular shape with a mean of its largest dimension being below 1000 micrometers, below 100 micrometers, and more preferably below 10 micrometers, and below 1 micrometer, and below 0.5 micrometer, and the deviation of the particles being less than 75% of the mean, less than 50% of the mean, less than 25% of the mean or less than 15% of the mean. In another aspect, the nanoparticle comprises the antigenic peptides or polypeptides, wherein the antigenic peptides or polypeptides are crosslinked, precipitating the antigenic peptides or polypeptides, aggregating the antigenic peptides or polypeptides, the particle is made of a different material such as metals or their oxides or their salts (gold, silver, iron oxide, aluminum hydroxide, aluminum phosphate), synthetic polymers (poly(lactide-co-glycolide), polycapralactone, polyanhydrides), inorganic molecules (silica), metal particles, zoonotic viruses, human viruses, bacterial viruses, plant viruses, bacteria, bacterial or fungal spores, yeast, liposomes, lipids, or other proteins and peptides that self-assemble, DNA/RNA molecules that self-assemble, pollen shells, carbohydrates, sugars, virus-like particles, or any combination of the aforementioned, a mixture of heterogenous particles made from one or more materials, the materials combined with a coating or a polymer layer on the metal particles, or by coating gold over silica particles, or combinations thereof. In another aspect, the antigenic peptides or polypeptides are mixed with particles, the antigenic peptides or polypeptides are chemically attached to a particle surface, or the antigenic peptides or polypeptides are entrapped in the particle core, or a combination thereof, wherein the antigenic peptides or polypeptides are in a free form and in an attached form. In another aspect, the one or more antigenic peptides or fusion polypeptides are made synthetically, recombinantly, in a prokaryotic expression system or a eukaryotic expression system. In another aspect, when two or more atoms or two or more molecules are included, they can be the same or different atoms or molecules. In another aspect, the atom is selected from any of the known elements of the periodic table. In another aspect, the atom is selected from gold or silver. In another aspect, the one or more molecules is one or more fat, one or more lipid, one or more carbohydrate, one or more natural or synthetic amino acid, one or more peptide, one or more protein, one or more nucleotide, one or more polymer synthetic or natural, or any combination thereof. In one aspect, the formulation further comprises an adjuvant selected from at least one of: monophosphoryl lipid A, synthetic lipid A, lipid A mimetics or analogs, aluminum salts, cytokines, saponins, muramyl dipeptide, N-glycolyl dipeptide, polyIC, polyCpG, lipopolysaccharide, polyphosphazenes, emulsions, virosomes, virus like particles, bacteria, algae, yeast, cochleates, poly(lactide-co-glycolides) microparticles, poloxamer particles, microparticles, toll-like receptor agonists, helper T cell agonists, T cell stimulating peptides added to the composition, T cell stimulating peptides conjugated or fused to the one or more antigenic peptides or fusion polypeptides, water in oil emulsion, oil in water emulsion, resiquimod, inulin, algammulin, lipid particles, or liposomes. In another aspect, the antigenic peptides or polypeptides are made synthetically or recombinantly. In another aspect, the composition is formulated for a mucosal, intranasal, intramuscular, intravenous, intrapulmonary, enteric, oral, subcutaneous, intradermal, subdermal, or transdermal route of administration. In another aspect, the peptide or polypeptide is expressed in a prokaryotic expression system or a eukaryotic expression system. In another aspect, the composition elicits immunoglobulin production, Th1 protective immunity, Th2 protective immunity, or any combination thereof. In another aspect, the formulation further comprises a buffer selected from the group consisting of phosphate buffer, citrate buffer, phosphate citrate buffer, borate buffer, tris(hydroxymethyl)aminomethane (Tris) containing buffer, succinate buffer, and buffers containing glycine or histidine as one of the buffering agents. In another aspect, the composition is in a liquid or a lyophilized form. In another aspect, the composition is contained within pre-filled syringes, microneedle patch, needle-free patch, and/or inhalation or nasal sprays. In another aspect, the one or more antigenic peptides or polypeptides comprise 2, 3, 4, 5, 6, 7, 8, 9, 10, 11, 12, 13, 14, 15, 16 or more antigenic peptides or polypeptides.

In another embodiment, the present invention includes a formulation comprising the molecule (A*ₙ₁B*ₙ₂C*ₙ₃D*ₙ₄E*ₙ₅F*ₙ₆G*ₙ₇H*ₙ₈*ₙ₉J*ₙ₁₀K*ₙ₁₁)ₙ₁₂, wherein A*, B*, C*, D*, E*, F*, G*, H*, I*, J*, K* are each a portion of contiguous amino acids selected from:

| Name | Protein | SEQ ID NO: |
|---|---|---|
| A | Spike_1 | 1 |
| B | Spike_2 | 2 |
| C | Spike_3 | 3 |
| D | Spike_4 | 4 |
| E | Spike_5 | 5 |
| F | Matrix_1 | 6 |
| G | NP_1 | 7 |
| H | RDRP_1 | 8 |
| I | RDRP_2 | 9 |
| J | RDRP_3 | 10 |
| K | RDRP_4 | 11 |

wherein n1, n2, n3, n4, n5, n6, n7, n8, n9, n10, and n11 can be any digit greater than or equal to zero but all are not simultaneously equal to zero, and the order of A*, B*, C*, D*, E*, F*, G*, H*, I*, J*, K* can be in any permutation and combination and n12 is greater than zero, wherein the peptides are optionally separated by a linker.

In another embodiment, the present invention includes a method of making an immunogenic composition comprising: selecting one or more antigenic peptides or fusion polypeptides from more than one strain of virus, wherein the one or more antigenic peptides or fusion polypeptides elicit at least one of a humoral, Th1, Th2 or CTL immune response; and conjugating the antigenic peptides or fusion polypeptides to a nanoparticle.

In another embodiment, the present invention includes a nucleic acid that encodes one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response. In one aspect, the nucleic acid is formulated into a composition is formulated into a vaccine for a mucosal, intranasal, intramuscular, intravenous, intrapulmonary, enteric, oral, subcutaneous, intradermal, subdermal, or transdermal route of administration. In another aspect, the one or more antigenic peptides or fusion polypeptides are expressed in a prokaryotic expression system or a eukaryotic expression system. In another aspect, the one or more antigenic peptides or fusion polypeptides are separated by a linker. In another aspect, the one or more antigenic peptides or fusion polypeptides are selected from at least one of SEQ ID NOS: 1 to 16, 22 to 39, any combination and concatemers thereof. In another aspect, the nucleic acid is formulated into a composition that elicits two or more immune responses selected from: immunoglobulin production, Th1 protective immunity, Th2 protective immunity, or any combination thereof. In another aspect, the vaccine is an RNA or a DNA vaccine.

In another embodiment, the present invention includes a host cell comprising a nucleic acid that encodes one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response. In another aspect, the vaccine is an RNA or a DNA vaccine.

In another embodiment, the present invention includes a nucleic acid expression vector that encodes one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response. In another aspect, the vaccine is an RNA or a DNA vaccine.

In another embodiment, the present invention includes a vaccine comprising nucleic acid that encodes one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response. In another aspect, the vaccine is an RNA or a DNA vaccine.

In another embodiment, the present invention includes a method of immunizing a subject comprising injecting the subject with an amount of a nucleic acid that encodes one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response, sufficient to trigger an immune response to the one or more antigenic peptides or fusion polypeptides. In another aspect, the vaccine is an RNA or a DNA vaccine.

### BRIEF DESCRIPTION OF THE DRAWINGS

For a more complete understanding of the features and advantages of the present invention, reference is now made to the detailed description of the invention along with the accompanying figures and in which:

FIGS. 1A to 1C show the results from Antigen 1 being conjugated to gold nanoparticles at different pH conditions. FIG. TA shows TEM images of gold nanoparticles (AuNP) before (upper images) and after the conjugation (lower images) with the fusion protein of Antigen 1 (CoVpep), under different conditions and amounts. FIG. 1B shows UV-Vis spectra of the AuNP before and after conjugation with CoVpep. FIG. 1C shows the diameter, zeta potential, and percent conjugation before and after conjugation of the AuNP and CoVpep.

FIG. 2A to 2D show the immunization schedule and results from the immunization of mice with the Antigen 1 of the present invention. FIG. 2A shows the immunization schedule and sample collection points. FIG. 2B includes four graphs (2A-A to 2A-D) that show the results from Anti-CoVpep serum antibody titration curve at day-42. FIG. 2C is a graph that shows anti-Spike protein serum antibody titration curve at day-42. FIG. 2D is a graph that shows anti-receptor binding domain (RBD) serum antibody titration curve at day-42.

FIGS. 3A to 3H show the cross-reactivity of serum antibodies against different strains of CoV at Day 0 and Day 42. FIG. 3A anti-SARS-CoV-2 IgG. FIG. 3B anti-SARS-CoV-1 IgG. FIG. 3C anti-MERS-CoV IgG. FIG. 3D anti-TEGV-CoV IgG. FIG. 3E anti-porcine respiratory-CoV IgG. FIG. 3F anti-canine-CoV IgG. FIG. 3G anti-avian infectious bronchitis-CoV IgG. FIG. 3H anti-Bovine-CoV-2 IgG.

FIGS. 4A to 4E show the antibody response, cross reactivity and virus neutralization from conserved spike pep1 peptide. Balb/c mice, 6-8 week old were vaccinated intramuscularly (IM) on day 0 and 21 with 40 μg pep1 conjugated on ˜60 μg AuNPs and 20 μg CpG. Day 42 serum was analyzed. (FIG. 4A) Sequence alignment of pep1 with different CoVs from alpha and beta genera (bolded), SEQ ID NOS:16, 16, 19, 20, and 21. (FIG. 4B) IgG, IgG1 and IgG2a antibody response towards pep1. (FIG. 4C) Neutralization of a pseudotyped reporter lentivirus containing SARS-CoV-2 spike protein on the surface and a GFP reporter gene. Mouse sera was heat inactivated at 56° C. for 30 min and added at two-fold dilutions (starting 1:20 dilution) to a fixed amount of reporter virus. After 1 h incubation the incubation mixture was added to confluent HEK293T cells expressing human ACE2 receptor in 96 well plates. After 72 h of culture the cells were run through FACS to determine percent of GFP expressing cells, which was then converted to % neutralization and reported. At low serum dilution (more antibodies) higher % neutralization is observed. (FIG. 4D) Live virus neutralization assay. Method from Poh et. al [71] was followed. Briefly, mouse sera was heat inactivated at 56° C. for 30 min and added at two-fold dilutions (starting 1:20 dilution) to a fixed amount (100 TCID50) of different strains of CoVs (human CoV-OC43, human CoV-NL63 and human CoV 229E). After 1 h incubation the incubation mixture was added to cell monolayers in 96 well plates (HCT-8 cells for human CoV-OC43, LLCMK2 cells for human CoV-NL63 and MRC-5 cells for human CoV-229E strains). After 7 days of culture incubation, the cell viability of each well was determined using Viral ToxGlo Assay (Promega, #G8941) to determine relative luminescence unit (RLU) using microplate reader (Biotek synergy H1). This RLU was then normalized and converted to % neutralization and reported. At low serum dilution (more antibodies) higher % neutralization is observed. (FIG. 4E) Infected-cell-based ELISA. Method from Conzelmann et. al [72] was followed. Briefly, cross reactivity of antibody response from pep1-based vaccine was assessed by measuring IgG binding to spike protein expressed in different live CoV infected cell lines. Confluent monolayers of HCT-8 cells, LLCMK2 cells, and MRC-5 cells were infected with human CoV-OC43, human CoV-NL63 and human CoV-229E strains, respectively, for 72 h, 72 h and 48 h incubation time at 34° C. and 5% CO2. Next, supernatant from each well was removed and cells were washed 3 times with PBS and fixed with 4% paraformaldehyde for 30 min. After fixation, the cells were washed and permeabilized with 0.1% Triton-X-100 for 5 min. After permeabilization, the cells were blocked with 3% BSA solution for 2 h at RT. After washing with PBST, the day 0 and day 42 immunized serum were incubated at 1:50 dilution for 1.5 h at room temperature (RT). The wells were then washed with PBST and incubated with HRP conjugated anti mouse IgG at 1:4000 dilution for 1.5 h at RT. After washing with PBST, OPD substrate was added for 15 min, and OD was measured at 492 nm.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

Vaccines are very effective for preventing and even eliminating infectious diseases. Although there are a number of efficacious vaccines based on full pathogens, development of safer, more potent and cost-effective vaccines based on portions of pathogen (subunit vaccines) is important. During the last two decades several approaches to the expression (bacterial, yeast, mammalian cell culture and plant) and delivery (DNA, live virus vectors, purified proteins, plant virus particles) of vaccine antigens have been developed. All these approaches have significant impact on the development and testing of newly developed candidate vaccines. There is a need for improving expression and delivery systems to create more efficacious and safer vaccines with fewer side effects. Highly desirable features of vaccines include: to be highly efficacious (stimulates both T and B cell immunity), to have a known and controlled composition, and that are easy to manufacture and purify.

### Definitions

As used herein, the term “antigen” refers to a molecule containing one or more epitopes (either linear, conformational or both) of the peptide(s) or protein(s) of Table 1 that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens. Thus, the antigenic peptide or antigenic polypeptide at least one type of, e.g., CoV, and preferably more than one type of CoV, or other virus as taught herein.

As used herein, the term “immunological response” refers to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the term an “immunogenic composition” refers to a composition that comprises an antigenic molecule that includes one or more of the peptide(s) or protein(s) of Table 1 formulated for administration to a subject that results in the development in the subject of a humoral and/or a cellular immune response to the peptide(s) or protein(s) of Table 1.

As used herein the terms “protein”, “polypeptide” or “peptide” refer to compounds comprising amino acids joined via peptide bonds and are used interchangeably. Alterations of the “protein”, “polypeptide” or “peptide” refer to those that have been changed by recombinant DNA engineering, chemical, or biochemical modifications, such as amino acid derivatives, amino acid or conjugates, post-translational modifications, or binding to a metal nanoparticle (such as a gold or silver nanoparticle), or a material that is coated with a metal.

As used herein, the term “fusion protein” refers to a hybrid protein, that includes portions of two or more different polypeptides, or fragments thereof, either synthesized chemically or resulting from the expression of a polynucleotide that encodes at least a portion of each of the two polypeptides.

As used herein, the term “substantially purified” refers to isolation of a substance (compound, polynucleotide, protein, polypeptide, polypeptide composition) such that the substance comprises the majority percent of the sample in which it resides. Typically, in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

As used herein, the term a “coding sequence” or a sequence which “encodes” a selected peptide(s) or protein(s) of Table 1, refers to a nucleic acid molecule that is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence. While in some cases it may be easier to synthesize the peptide(s) or protein(s) of Table 1 from individual amino acids, it is also possible to create a coding sequence that is under the control of an expression promoter that is used to express the peptide(s) or protein(s) of Table 1 in a cell, e.g., a bacterial, yeast, insect, mammalian or plant cell.

As used herein, the term “control elements”, includes, but is not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences, and/or sequence elements controlling an open chromatin structure see e.g., McCaughan et al. (1995) PNAS USA 92:5431-5435; Kochetov et al (1998) FEBS Letts. 440:351-355.

As used herein, the term “nucleic acid” includes, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also captures sequences that include any of the known base analogs of DNA and RNA.

As used herein, the term “operably linked” refers to an arrangement of elements wherein the components so described are configured so as to perform their usual function. Thus, a given promoter operably linked to a coding sequence is capable of effecting the expression of the coding sequence when active. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

As used herein, the term “recombinant” refers to a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

Techniques for determining amino acid sequence “similarity” are well known in the art. In general, “similarity” means the exact amino acid to amino acid comparison of two or more polypeptides at the appropriate place, where amino acids are identical or possess similar chemical and/or physical properties such as charge or hydrophobicity. A so-termed “percent similarity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby, and comparing this to a second amino acid sequence. In general, “identity” refers to an exact nucleotide to nucleotide or amino acid to amino acid correspondence of two polynucleotides or polypeptide sequences, respectively.

As used herein, the term a “vector” refers to a nucleic acid capable of transferring gene sequences to target cells (e.g., bacterial plasmid vectors, viral vectors, non-viral vectors, particulate carriers, and liposomes). Typically, “vector construct,” “expression vector,” and “gene transfer vector,” mean any nucleic acid construct capable of directing the expression of one or more sequences of interest in a host cell. Thus, the term includes cloning and expression vehicles, as well as viral vectors. The term is used interchangeable with the terms “nucleic acid expression vector” and “expression cassette.”

Many suitable expression or manufacturing systems are commercially available, including, for example, the following: Ausubel, F. M., et al., CURRENT PROTOCOLS IN MOLECULAR BIOLOGY, John Wiley and Sons, Inc., Media Pa.; Clontech), Goeddel, D. V., Methods in Enzymology 185 (1990); Guthrie, C., and G. R. Fink, Methods in Enzymology 194 (1991)), relevant portions incorporated herein by reference.

As used herein, the term “subject” refers to any chordates, including, but not limited to, humans and other primates, bats, non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

As used herein, the term “treatment” refers to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection).

As used herein, the term “adjuvant” refers to a substance that non-specifically changes or enhances an antigen-specific immune response of an organism to the antigen. Generally, adjuvants are non-toxic, have high-purity, are degradable, and are stable. The recombinant adjuvant of the present invention meets all of these requirements; it is non-toxic, highly-pure, degradable, and stable. Adjuvants are often included as one component in a vaccine or therapeutic composition that increases the specific immune response to the antigen. Non-limiting examples of adjuvants for use with the present invention include at least one of: monophosphoryl lipid A, synthetic lipid A, lipid A mimetics or analogs, aluminum salts, cytokines, saponins, muramyl dipeptide, N-glycolyl dipeptide, polyIC, polyCpG, lipopolysaccharide, polyphosphazenes, emulsions, virosomes, virus like particles, bacteria, algae, yeast, cochleates, poly(lactide-co-glycolides) microparticles, poloxamer particles, microparticles, toll-like receptor agonists, helper T cell agonists, T cell stimulating peptides added to the composition, T cell stimulating peptides conjugated or fused to the one or more antigenic peptides or fusion polypeptides, water in oil emulsion, oil in water emulsion, resiquimod, inulin, algammulin, lipid particles, or liposomes.

As used herein, the terms “effective dose”, “effective amount” or “amount effective to” refer to that amount of an immunogenic fusion peptide(s) or protein(s) of Table 1, or combinations thereof, provided in an amount that is sufficient to induce immunity, to prevent and/or ameliorate an infection or to reduce at least one symptom of an infection, and/or to enhance the efficacy of a dose of the immunogenic fusion peptide(s) or protein(s) of Table 1, or combinations thereof against a coronavirus in a mammal or avian. An effective dose may refer to the amount of the fusion peptide or protein sufficient to delay or minimize the onset of an infection. An effective dose may also refer to the fusion protein peptide(s) or protein(s) of Table 1 in an amount that provides a therapeutic benefit in the treatment or management of an infection. Further, an effective dose is the amount with respect to the peptide(s) or protein(s) of Table 1, alone or in combination with other therapies, that provide(s) a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

As used herein, the term “multivalent” refers to fusion proteins that have multiple antigenic peptide(s) or protein(s) of Table 1 against multiple types or strains of CoV.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as the peptide(s) or protein(s) of Table 1 of the present invention or can be administered separately. Either the protein or an expression vector encoding the protein can be administered to produce an immunostimulatory effect.

As used herein, the term “innate immune response stimulator” refers to agents that trigger the innate or non-specific immune response. The innate immune response is a nonspecific defense mechanism is able to act immediately (or within hours) of an antigen's appearance in the body and the response to which is non-specific, that is, it responds to an entire class of agents (such as oligosaccharides, lipopolysaccharides, nucleic acids such as the CpG motif, etc.) and does not generate an adaptive response, that is, they do not cause immune memory to the antigen. Pathogen-associated immune stimulants act through the Complement cascade, Toll-like Receptors, and other membrane bound receptors to trigger phagocytes to directly kill the perceived pathogen via phagocytosis and/or the expression of immune cell stimulating cytokines and chemokines to stimulate both the innate and adaptive immune responses.

As used herein, the term “protective immune response” or “protective response” refers to an immune response mediated by antibodies or effector cells against an infectious agent, which is exhibited by a vertebrate (e.g., a human), which prevents or ameliorates an infection or reduces at least one symptom thereof. The peptide(s) or protein(s) of Table 1 of the invention can stimulate the production of antibodies that, for example, neutralize infectious agents, blocks infectious agents from entering cells, blocks replication of said infectious agents, and/or protect host cells from infection and destruction. The term can also refer to an immune response that is mediated by T-lymphocytes and/or other white blood cells against an infectious agent, exhibited by a vertebrate (e.g., a human), that prevents or ameliorates coronavirus infection or reduces at least one symptom thereof.

As used herein, the term “antigenic formulation” or “antigenic composition” refers to a preparation which, when administered to a vertebrate, e.g. a mammal, will induce an immune response.

As used herein, the terms “immunization” or “vaccine” are used interchangeably to refer to a formulation that contains the fusion protein(s) of the present invention, which is in a form that is capable of being administered to a vertebrate and which induces a protective immune response sufficient to induce immunity to prevent and/or ameliorate an infection and/or to reduce at least one symptom of an infection and/or to enhance the efficacy of another dose or exposure to the coronavirus. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

The practice of the present invention employs, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

Coronaviruses (CoVs) belong to the family Coronaviridae and, as the name ‘corona’ indicates, CoVs have a characteristic crown-like appearance on their outer surface due to the spike protein. The spike protein facilitates the entry of the virus into host cells. The CoV family is comprised of four genera: alpha, beta, gamma, and delta CoVs. Alpha- and beta-CoVs infect diverse species such as mammals, cats, bats, mice, pigs, and humans. Gamma and delta-CoVs generally infect birds, but a few can also infect mammals. Such a broad diversity of infectivity and reservoir species greatly increases the chance of the CoV transferring from animals and birds to humans, which been the case for the three CoV outbreak within the last 18 years. As such, what is needed are vaccination (immunizations) that can be used to provide widespread protection against existing and emerging CoV, that are easy to mass produce, and that can be adapted and adjusted to the most recent or newly recognized CoV.

Different coronaviruses have infected humans so far. So far, seven CoVs, including the 2019 pandemic strain have been known to infect humans. The human coronaviruses (HCoVs) belong to two of these genera, namely the alpha coronaviruses (includes HCoV-229E and HCoV-NL63)[13, 14] and beta coronaviruses (includes HCoV-HKU1, HCoV-OC43, the severe acute respiratory syndrome coronavirus: SARS-CoV, Middle East respiratory syndrome coronavirus: MERS-CoV, and the novel corona virus from 2019: 2019-nCoV or SAR-CoV-2)[15-22]. Amongst these, three strains have caused severe infections in humans; SARS-CoV (˜10% mortality rate) originated from China in 2002[15], MERS-CoV (˜34.4% mortality rate) originated from Saudi Arabia in 2012[18], and the newly identified 2019-nCoV SAR-CoV2 (˜4.4% mortality rate) [23] originated from Wuhan, China in December 2019[21, 24], which has now become a worldwide pandemic. Until Jul. 14, 2020, pandemic 2019-nCoV has infected more than 13 million people worldwide and has led to 576,752 deaths, and continues to pose significant threat to global public health. The origin of SARS-CoV and MERS-CoV is thought to be bats. Genome sequences of 2019-nCoV match 79.5% and 96% similarity at nucleotide level to the SARS-CoV and bat CoVs, respectively, which suggests that the nCoV also originated in bats.

Coronavirus genome expresses multiple proteins. The CoV is an enveloped, positive-sense, single-stranded RNA virus. The RNA strand is 27 to 32 kb long, making it the largest virus RNA genome. Since the mutation rates of RNA viruses are higher than DNA viruses, CoVs can readily adapt for infection and survival. Although the genome of different genera of CoVs is slightly different but all CoVs code for four main structural proteins, namely the spike (S), nucleocapsid (NP), envelope (E), and membrane (M) proteins on the surface of virus, and other non-structural proteins like RNA directed RNA polymerase, 3CL like proteinases, helicase and 15-16 nonstructural proteins that are produced by the cleavage of ORF lab[25]. ORF lab occupies two thirds of the viral genome at the 5′ end of genome, whereas the S, E, M and NP proteins represent a third of genome at the 3′ end. Most of the nonstructural proteins participates in virus replication and transcription[26], but some also help the virus to evade the immune system[27]. The S protein is made up of S1 and S2 subunits, S1 subunit helps in virus-host cell receptor binding and S2 subunit help in virus-host membrane fusion[28, 29]. S1 subunit is further divided into C-terminal domain (CTD) and N-terminal domain (NTD). The combined CTD and NTD is known as receptor-binding domain (RBD)[30, 31]. RBD, has the ability to recognize host receptor, which is able to initiate S protein conformational change and subsequently the virus membrane fuses with host cell using the S2 subunit [32, 33]. To add to the complexity, different CoVs use different host receptors, e.g., HCoV-229E uses aminopeptidase N (APN)[34], HCoV-OC43 and HCoV-HKU1 utilize sialic acid[35], HCoV-NL63, SARS-CoV and 2019-nCoV use angiotensin-converting enzyme 2 (ACE2)[36, 37], and MERS-CoV uses dipeptidyl peptidase 4 (DPP4)[38] to enter the cell. Thus, CoVs are a highly diverse and well-adapted virus family.

The Coronavirus genome can readily mutate resulting in new strains, thus, the current vaccine design relying on the spike surface protein as the target antigen limits vaccine efficacy. Bats have been discovered to be the reservoir for CoVs. A bat population in a cave in China was found to contain a large diversity of CoVs genes. Importantly, gene fragments from this diverse set of CoVs genes, can recombine to generate the entirely new genome of the SARS-CoV[6]. Such caves have now come to be regarded as ‘hot-spots’ of CoVs from where, due to naturally high mutation rates of RNA viruses in conjunction with frequent recombination of genomes of different CoVs coinfecting the same host, a novel CoV can emerge that is highly pathogenic and easily transmittable to humans. As such, due to this high inherent diversity on CoVs, they remain a moving target.

Current vaccines rely on the use of the spike surface protein of the coronavirus as an antigen. The antigen is either delivered in the form of a protein or mRNA/DNA that expresses the spike protein. Using this approach, the vaccine-induced protection is directed towards one type of coronavirus. It has been seen that different spike proteins target different cell receptors to gain entry into cells. Thus, the approach is limited because if a new coronavirus were to emerge, then a new vaccine will be needed. Making a new vaccine takes time, often a couple of years even at the fastest speed. Therefore, there is a need to make a vaccine that can offer broader protection against multiple coronaviruses.

Design of a vaccine with broad protection. This invention discloses antigen sequences that are conserved in different coronaviruses (human, avian and zoonotic coronaviruses). The inventors have performed a protein sequence analysis of all seven human CoVs (HCoVs), which have circulated in and infected the human population so far. These include HCoV-229E, HCoV-OC43, HCoV-NL63, HCoV-HKU1 SARS-CoV, MERS-CoV and 2019-nCoV. The inventors retrieved complete protein sequences of spike, envelope, membrane, nucleocapsid and replicase polyprotein lab or ORF lab polyprotein from NCBI (https://www.ncbi.nlm.nih.gov/labs/virus/vssi/#/) online server, multiple aligned them using Clustal omega (https://www.ebi.ac.uk/Tools/msa/clustalo/) online server, and alignment file was used in EMBOSS (https://www.ebi.ac.uk/Tools/msa/emboss_cons/) to identify consensus sequence of each human CoVs. The inventors identified 11 conserved peptides to target different region of human coronavirus including recently emerged 2019-nCoV. The inventors found 5 conserved peptides region from spike protein, 4 from RNA dependent RNA polymerase region and 1 for each matrix and nucleoprotein region. These conserved regions were also examined for putative T and B cell epitopes using the NIAID Immune Epitope Database (IEDB). Each peptide was seen to have B or T cell epitopes or both. The conserved sequences and epitopes are shown in Table 1.

TABLE 1
Conserved sequences and location in corona virus protein. These conserved
regions were used for T and B cell epitope search by IEDB database.
Epitope sequence denoted in Bold are B-cell epitopes; in Underline
are T-cell epitopes, and in Italics are regions that include both
B and T cell epitopes, which are SEQ ID NOS 1-11, respectively.

| | | | SEQ |
|---|---|---|---|
| | | | ID |
| ID | Conserved Sequence | Conservation | NO: |
| Spike _1 | RSFIEDLLFNKVTLADAG | 98% in COVID-19, 95% in SARS, 66% in MERS, | 1 |
| | FMKQYGDCLGDIAARDLI | >56% in HCoV-OC43 and HCoV-HKU1, >38% in | |
| | CAQKF | HCoV-229E and HCoV-NL63 and >40% in most | |
| | | other animal Corona viruses | |
| Spike_2 | YRFNGIGVTQNVLYENQK | 100% in COVID-19, 95% in SARS, 78% in | 2 |
| | LIAN | MERS, 73% in HCoV-OC43, 72% in HCoV- | |
| | | HKU1, 50% in HCoV-229E, 63% in HCoV-NL63 | |
| | | and >50% in most other animal Corona viruses | |
| Spike_3 | KLQDVVNQNAQALN | 100% in COVID-19 and SARS, 78% in MERS, | 3 |
| | | 72% in HCoV-OC43 and HCoV-229E, 79% in | |
| | | HCoV-HKU1, >65% in HCoV-NL-63 and >50% | |
| | | in most other animal Corona viruses | |
| Spike_4 | QLSSNFGAISSVLNDILSR | 100% in COVID-19 and SARS, 60% in MERS, | 4 |
| | LDKVEAEVQIDRLITGRL | 73% in HCoV-OC43 and HCoV-HKU1, 60% in | |
| | | HCOV-229E, 57% in HCoV-NL63 and >50% in | |
| | | most other animal Corona viruses | |
| Spike_5 | YIKWPWYIWL | 100% in COVID-19, 90% in SARS and MERS, | 5 |
| | | 80% in HCoV-OC43, HCoV-HKU1, HCoV-229E, | |
| | | HCoV-NL63 and >75% in most other animal | |
| | | Corona viruses | |
| Matrix_1 | MWLSYFIASFRLFARTRS | 100% in COVID-19, 96% in SARS, 74% in | 6 |
| | MWSFNPETN | MERS, 71% in HCoV-OC43, 70% in HCoV- | |
| | | HKU1, 60% in HCoV-229E, 57% in HCoV-NL63 | |
| | | and >50% in most other animal Corona viruses | |
| NP_1 | PRWYFYYLGTGP | 100% in All Human Coronavirus except 92% in | 7 |
| | | MERS-COV and 66% in HCoV-229E, HCoV- | |
| | | NL63 | |
| RDRP_1 | VGVLTLDNQDL | 100% in All Human Coronavirus and most of | 8 |
| | | animal Corona viruses^{#} | |
| RDRP_2 | LMGWDYPKCDRA | 100% in All Human Coronavirus and most of | 9 |
| | | animal Corona viruses* | |
| RDRP_3 | KHFSMMILSDD | 100% in All Human Corona virus | 10 |
| RDRP_4 | TQMNLKYAISAKNRARTVA | 100% in All Human Coronavirus except 82% in | 11 |
| | GVS | 229E and NL-63 strain | |

Below are shown four examples of polypeptide sequence designs, which use peptides of Table 1 and link to each or the other with or without different linkers, which are SEQ ID NOS 12-15, respectively. These combination molecules can be recombinantly or synthetically synthesized and used directly as large peptide molecules, or they can be used as DNA/mRNA vaccines, or with particles and/or adjuvant.

SEQ ID NO:12 was created from sequences in Table 1 according to the structure: KKKK-(RDRP_1)₅-KKKK-(RDRP_2)₅-KKKK-(RDRP_3)₅-KKKK-(RDRP_4)₅-KKKK(Spike_1)*₅-KKKK-(NP_1)*₅-C^{#}; where the numerical digit on the subscript indicates the number of times the sequence ID is repeated, KKKK is the linker (SEQ ID NO:40), * indicates that only a portion of the sequence listed in Table 1 is used, and C^{#} is a thiol containing amino acid, the RDRP sequences are in Table 1 and are SEQ ID NOS: 8-11.

(SEQ ID NO: 12)
KKKKVGVLTLDNQDLVGVLTLDNQDLVGVLTLDNQDLVGVLTLDNQDLV
GVLTLDNQDLKKKKLMGWDYPKCDRALMGWDYPKCDRALMGWDYPKCDR
ALMGWDYPKCDRALMGWDYPKCDRAKKKKHFSMMILSDDKHFSMMILSD
DKHFSMMILSDDKHFSMMILSDDKHFSMMILSDDKKKKKTQMNLKYAIS
AKNRARTVAGVSTQMNLKYAISAKNRARTVAGVSTQMNLKYAISAKNRA
RTVAGVSTQMNLKYAISAKNRARTVAGVSTQMNLKYAISAKNRARTVAG
VSKKKKSFIEDLLFNKVSFIEDLLFNKVSFIEDLLFNKVSFIEDLLFNK
VSFIEDLLFNKVKKKKFYYLGTGPFYYLGTGPFYYLGTGPFYYLGTGPF
YYLGTGPC^{#}

SEQ ID NO:13 is created from sequences in Table 1 according to the structure: (Spike_1)₅-(Spike_2)₅-(Spike_3)₅-(Spike_4)₅-(Spike_5)₅-(Matrix_1)₅-(RDRP_1)₅-(RDRP_2)₅-(RDRP_3)₅-(RDRP_4)₅-(NP_1)₅-C^{#}; where the numerical digit on the subscript indicates the number of times the sequence ID is repeated, and C^{#} is a thiol containing amino acid, the peptide sequences are alternatively underlined and not underlined.

(SEQ ID NO: 13)
RSFIEDLLFNKVTLADAGFMKQYGDCLGDIAARDLICAQKFRSFIEDLL
FNKVTLADAGFMKQYGDCLGDIAARDLICAQKFRSFIEDLLFNKVTLAD
AGFMKQYGDCLGDIAARDLICAQKFRSFIEDLLFNKVTLADAGFMKQYG
DCLGDIAARDLICAQKFRSFIEDLLFNKVTLADAGFMKQYGDCLGDIAA
RDLICAQKFYRFNGIGVTQNVLYENQKLIANYRFNGIGVTQNVLYENQK
LIANYRFNGIGVTQNVLYENQKLIANYRFNGIGVTQNVLYENQKLIANY
RFNGIGVTQNVLYENQKLIANKLQDVVNQNAQALNKLQDVVNQNAQALN
KLQDVVNQNAQALNKLQDVVNQNAQALNKLQDVVNQNAQALNQLSSNFG
AISSVLNDILSRLDKVEAEVQIDRLITGRLQLSSNFGAISSVLNDILSR
LDKVEAEVQIDRLITGRLQLSSNFGAISSVLNDILSRLDKVEAEVQIDR
LITGRLQLSSNFGAISSVLNDILSRLDKVEAEVQIDRLITGRLQLSSNF
GAISSVLNDILSRLDKVEAEVQIDRLITGRLYIKWPWYIWLYIKWPWYI
WLYIKWPWYIWLYIKWPWYIWLYIKWPWYIWLMWLSYFIASFRLFARTR
SMWSFNPETNMWLSYFIASFRLFARTRSMWSFNPETNMWLSYFIASFRL
FARTRSMWSFNPETNMWLSYFIASFRLFARTRSMWSFNPETNMWLSYFI
ASFRLFARTRSMWSFNPETNVGVLTLDNQDLVGVLTLDNQDLVGVLTLD
NQDLVGVLTLDNQDLVGVLTLDNQDLLMGWDYPKCDRALMGWDYPKCDR
ALMGWDYPKCDRALMGWDYPKCDRALMGWDYPKCDRAKHFSMMILSDDK
HFSMMILSDDKHFSMMILSDDKHFSMMILSDDKHFSMMILSDDTQMNLK
YAISAKNRARTVAGVSTQMNLKYAISAKNRARTVAGVSTQMNLKYAISA
KNRARTVAGVSTQMNLKYAISAKNRARTVAGVSTQMNLKYAISAKNRAR
TVAGVSPRWYFYYLGTGPPRWYFYYLGTGPPRWYFYYLGTGPPRWYFYY
LGTGPPRWYFYYLGTGPC^{#}

3. SEQ ID NO:14 is created from sequences in Table 1 according to the structure: (Spike_1)-GGDGG-(Spike_1)-GGDGG-(Spike_1)-GGDGG-(Spike_1)-GGDGG-(Spike_1)-GGDGG-(Spike_2)-GGDGG-(Spike_2)-GGDGG-(Spike_2)-GGDGG-(Spike_2)-GGDGG-(Spike_2)-GGDGG-(Spike_3)-GGDGG-(Spike_3)-GGDGG-(Spike_3)-GGDGG-(Spike_3)-GGDGG-(Spike_3)-GGDGG-(Spike_4)-GGDGG-(Spike_4)-GGDGG-(Spike_4)-GGDGG-(Spike_4)-GGDGG-(Spike_4)-GGDGG-(Spike_5)-GGDGG-(Spike_5)-GGDGG-(Spike_5)-GGDGG-(Spike_5)-GGDGG-(Spike_5)-GGDGG-(Matrix_1)-GGDGG-(Matrix_1)-GGDGG-(Matrix_1)-GGDGG-(Matrix_1)-GGDGG-(Matrix_1)-GGDGG-(RDPP_1)-GGDGG-(RDRP_1)-GGDGG-(RDRP_1)-GGDGG-(RDRP_1)-GGDGG-(RDRP_1)-GGDGG-(RDRP_2)-GGDGG-(RDRP_2)-GGDGG-(RDRP_2)-GGDGG-(RDRP_2)-GGDGG-(RDRP_2)-GGDGG-(RDRP_3)-GGDGG-(RDRP_3)-GGDGG-(RDRP_3)-GGDGG-(RDRP_3)-GGDGG-(RDRP_3)-GGDGG-(RDRP_4)-GGDGG-(RDRP_4)-GGDGG-(RDRP_4)-GGDGG-(RDRP_4)-GGDGG-(RDRP_4)-GGDGG-(NP_1)-GGDGG-(NP_1)-GGDGG-(NP_1)-GGDGG-(NP_1)-GGDGG-(NP_1)-C^{#}; where GGDGG is the linker and is SEQ ID NO:41, and C is a thiol containing amino acid, the spike, matrix, and RDPR sequences are in Table 1.

(SEQ ID NO: 14)
RSFIEDLLFNKVTLADAGFMKQYGDCLGDIAARDLICAQKFGGDGGRSF
IEDLLFNKVTLADAGFMKQYGDCLGDIAARDLICAQKFGGDGGRSFIED
LLFNKVTLADAGFMKQYGDCLGDIAARDLICAQKFGGDGGRSFIEDLLF
NKVTLADAGFMKQYGDCLGDIAARDLICAQKFGGDGGRSFIEDLLFNKV
TLADAGFMKQYGDCLGDIAARDLICAQKFGGDGGYRFNGIGVTQNVLYE
NQKLIANGGDGGYRFNGIGVTQNVLYENQKLIANGGDGGYRFNGIGVTQ
NVLYENQKLIANGGDGGYRFNGIGVTQNVLYENQKLIANGGDGGYRFNG
IGVTQNVLYENQKLIANGGDGGKLQDVVNQNAQALNGGDGGKLQDVVNQ
NAQALNGGDGGKLQDVVNQNAQALNGGDGGKLQDVVNQNAQALNGGDGG
KLQDVVNQNAQALNGGDGGQLSSNFGAISSVLNDILSRLDKVEAEVQID
RLITGRLGGDGGQLSSNFGAISSVLNDILSRLDKVEAEVQIDRLITGRL
GGDGGQLSSNFGAISSVLNDILSRLDKVEAEVQIDRLITGRLGGDGGQL
SSNFGAISSVLNDILSRLDKVEAEVQIDRLITGRLGGDGGQLSSNFGAI
SSVLNDILSRLDKVEAEVQIDRLITGRLGGDGGYIKWPWYIWLGGDGGY
IKWPWYIWLGGDGGYIKWPWYIWLGGDGGYIKWPWYIWLGGDGGYIKWP
WYIWLGGDGGMWLSYFIASFRLFARTRSMWSFNPETNGGDGGMWLSYFI
ASFRLFARTRSMWSFNPETNGGDGGMWLSYFIASFRLFARTRSMWSFNP
ETNGGDGGMWLSYFIASFRLFARTRSMWSFNPETNGGDGGMWLSYFIAS
FRLFARTRSMWSFNPETNGGDGGVGVLTLDNQDLGGDGGVGVLTLDNQD
LGGDGGVGVLTLDNQDLGGDGGVGVLTLDNQDLGGDGGVGVLTLDNQDL
GGDGGLMGWDYPKCDRAGGDGGLMGWDYPKCDRAGGDGGLMGWDYPKCD
RAGGDGGLMGWDYPKCDRAGGDGGLMGWDYPKCDRAGGDGGKHFSMMIL
SDDGGDGGKHFSMMILSDDGGDGGKHFSMMILSDDGGDGGKHFSMMILS
DDGGDGGKHFSMMILSDDGGDGGTQMNLKYAISAKNRARTVAGVSGGDG
GTQMNLKYAISAKNRARTVAGVSGGDGGTQMNLKYAISAKNRARTVAGV
SGGDGGTQMNLKYAISAKNRARTVAGVSGGDGGTQMNLKYAISAKNRAR
TVAGVSGGDGGPRWYFYYLGTGPGGDGGPRWYFYYLGTGPGGDGGPRWY
FYYLGTGPGGDGGPRWYFYYLGTGPGGDGGPRWYFYYLGTGPC^{#}

SEQ ID NO:15 is created from sequences in Table 1 according to the structure: (Spike_1)-GPGPG-(Spike_1)-GPGPG-(Spike_1)-GPGPG-(Spike_1)-GPGPG-(Spike_1)-GPGPG-(Spike_2)-GPGPG-(Spike_2)-GPGPG-(Spike_2)-GPGPG-(Spike_2)-GPGPG-(Spike_2)-GPGPG-(Spike_3)-GPGPG-(Spike_3)-GPGPG-(Spike_3)-GPGPG-(Spike_3)-GPGPG-(Spike_3)-GPGPG-(Spike_4)-GPGPG-(Spike_4)-GPGPG-(Spike_4)-GPGPG-(Spike_4)-GPGPG-(Spike_4)-GPGPG-(Spike_5)-GPGPG-(Spike_5)-GPGPG-(Spike_5)-GPGPG-(Spike_5)-GPGPG-(Spike_5)-GPGPG-(Matrix_1)-GPGPG-(Matrix_1)-GPGPG-(Matrix_1)-GPGPG-(Matrix_1)-GPGPG-(Matrix_1)-GPGPG-(RDPP_1)-GPGPG-(RDRP_1)-GPGPG-(RDRP_1)-GPGPG-(RDRP_1)-GPGPG-(RDRP_1)-GPGPG-(RDRP_2)-GPGPG-(RDRP_2)-GPGPG-(RDRP_2)-GPGPG-(RDRP_2)-GPGPG-(RDRP_2)-GPGPG-(RDRP_3)-GPGPG-(RDRP_3)-GPGPG-(RDRP_3)-GPGPG-(RDRP_3)-GPGPG-(RDRP_3)-GPGPG-(RDRP_4)-GPGPG-(RDRP_4)-GPGPG-(RDRP_4)-GPGPG-(RDRP_4)-GPGPG-(RDRP_4)-GPGPG-(NP_1)-GPGPG-(NP_1)-GPGPG-(NP_1)-GPGPG-(NP_1)-GPGPG-(NP_1)-C^{#}; where GPGPG is the linker and has SEQ ID NO:42, and C^{#} is a thiol containing amino acid, the spike, matrix, and RDPR sequences are in Table 1.

(SEQ ID NO: 15)
RSFIEDLLFNKVTLADAGFMKQYGDCLGDIAARDLICAQKFGPGPGRSF
IEDLLFNKVTLADAGFMKQYGDCLGDIAARDLICAQKFGPGPGRSFIED
LLFNKVTLADAGFMKQYGDCLGDIAARDLICAQKFGPGPGRSFIEDLLF
NKVTLADAGFMKQYGDCLGDIAARDLICAQKFGPGPGRSFIEDLLFNKV
TLADAGFMKQYGDCLGDIAARDLICAQKFGPGPGYRFNGIGVTQNVLYE
NQKLIANGPGPGYRFNGIGVTQNVLYENQKLIANGPGPGYRFNGIGVTQ
NVLYENQKLIANGPGPGYRFNGIGVTQNVLYENQKLIANGPGPGYRFNG
IGVTQNVLYENQKLIANGPGPGKLQDVVNQNAQALNGPGPGKLQDVVNQ
NAQALNGPGPGKLQDVVNQNAQALNGPGPGKLQDVVNQNAQALNGPGPG
KLQDVVNQNAQALNGPGPGQLSSNFGAISSVLNDILSRLDKVEAEVQID
RLITGRLGPGPGQLSSNFGAISSVLNDILSRLDKVEAEVQIDRLITGRL
GPGPGQLSSNFGAISSVLNDILSRLDKVEAEVQIDRLITGRLGPGPGQL
SSNFGAISSVLNDILSRLDKVEAEVQIDRLITGRLGPGPGQLSSNFGAI
SSVLNDILSRLDKVEAEVQIDRLITGRLGPGPGYIKWPWYIWLGPGPGY
IKWPWYIWLGPGPGYIKWPWYIWLGPGPGYIKWPWYIWLGPGPGYIKWP
WYIWLGPGPGMWLSYFIASFRLFARTRSMWSFNPETNGPGPGMWLSYFI
ASFRLFARTRSMWSFNPETNGPGPGMWLSYFIASFRLFARTRSMWSFNP
ETNGPGPGMWLSYFIASFRLFARTRSMWSFNPETNGPGPGMWLSYFIAS
FRLFARTRSMWSFNPETNGPGPGVGVLTLDNQDLGPGPGVGVLTLDNQD
LGPGPGVGVLTLDNQDLGPGPGVGVLTLDNQDLGPGPGVGVLTLDNQDL
GPGPGLMGWDYPKCDRAGPGPGLMGWDYPKCDRAGPGPGLMGWDYPKCD
RAGPGPGLMGWDYPKCDRAGPGPGLMGWDYPKCDRAGPGPGKHFSMMIL
SDDGPGPGKHFSMMILSDDGPGPGKHFSMMILSDDGPGPGKHFSMMILS
DDGPGPGKHFSMMILSDDGPGPGTQMNLKYAISAKNRARTVAGVSGPGP
GTQMNLKYAISAKNRARTVAGVSGPGPGTQMNLKYAISAKNRARTVAGV
SGPGPGTQMNLKYAISAKNRARTVAGVSGPGPGTQMNLKYAISAKNRAR
TVAGVSGPGPGPRWYFYYLGTGPGPGPGPRWYFYYLGTGPGPGPGPRWY
FYYLGTGPGPGPGPRWYFYYLGTGPGPGPGPRWYFYYLGTGPC*

It is recognized that vaccines based on protein-subunits and short peptides are not highly immunogenic. There are several ways of enhancing their immune responses such as the ones described in references: https://www.frontiersin.org/articles/10.3389/fimmu.2018.02224/full, www.nature.com/articles/s41565-020-0737-y, www.sciencedirect.com/science/article/pii/S181808761400035X, pubs.rsc.org/en/content/articlelanding/2020/nr/c9nr08958f#!divAbstract, www.nature.com/articles/s41565-020-0737-y, www.ncbi.nlm.nih.gov/pmc/articles/PMC22232/, www.futuremedicine.com/doi/10.2217/nnm-2018-0147, pubmed.ncbi.nlm.nih.gov/23829488/, https://pubmed.ncbi.nlm.nih.gov/25842219/). These references are incorporated in their entirety but do not limit the scope of this invention. Some of the approaches to enhance the immunogenicity of the protein subunit and peptide vaccines rely on converting the vaccine into a particle format. The particles can be prepared in many ways and all these approaches that render a protein or peptide molecule into a particle formulation are incorporated in this invention by reference. Other ways to enhance the immune response of the host includes the use of adjuvants and immunomodulators or targeting the vaccine formulation to specific immune cells or tissues such as lymph nodes and the skin.

Experimental Data.

A portion of the peptide labeled Spike_1 in Table 1 was synthesized as per the following design.

An example of the present invention is shown as Antigen 1, which includes, in bold letters (aka COVpep fusion peptide in figure set), the linker is underlined and in italics is a sulfur containing amino acid used to link the peptide(s) to a gold particle:

Antigen 1—SFIEDLLFNKVTLADAGFKKKKC (SEQ ID NO:16).

SFIEDLLFNKVTLADAGF is a portion of spike_1 (see Table 1) (SEQ ID NO: 17).

KKKK is the linker to make the peptide more hydrophilic (SEQ ID NO: 40).

C is amino acid cysteine added to help attach the peptide to gold nanoparticles (AuNPs). Alternatively, the fusion protein can include one or more cysteines, but can also include non-natural or alternative amino acids, such as selenocysteine.

C is amino acid cysteine added to help attach the peptide to gold nanoparticles (AuNPs). Alternatively, the fusion protein can include one or more cysteines, but can also include non-natural or alternative amino acids, such as selenocysteine.

Conjugation of CoVpep on the surface of gold nanoparticle at different pH. AuNPs of 12 nm diameter were chemically synthesized by the Turkevich method. Nanoparticle suspension (1.5 ml) was mixed with tween 20 (0.1%) in different microcentrifuge tubes. All microcentrifuge tubes were centrifuged at 17,000 g for 25 min at 4° C. to pellet the AuNPs. The supernatant was removed and water containing tween 20 (0.1%) at pH ranging from 5 to 11 was used to wash the AuNPs three times. The same water at respective pH was also used to make the CoVpep stock (5 mg/ml). After three washings, pellets of AuNPs were resuspended in 96 μl (for the 40 μg CoVpep/mouse dose) or 120 μl (for the 20 μg CoVpep/mouse dose) of water at respective pH (range 5-11). To achieve 40 μg CoVpep/mouse dose, 48 μl of 5 mg/ml CoVpep in water at respective pH (range 5-11) was added to 96 μl of the AuNP suspension. For 20 μg CoVpep/mouse dose, 24 μl (equivalent to 120 μg) of 5 mg/ml CoVpep in water at respective pH (range 5-11) was added to 120 μl of the AuNP suspension (total 336 μg). Both formulation (total volume 150 μl) at different Ph conditions (range 5-11) were incubated overnight (around 12-14h) at 4° C. Now, these formulations are ready for immunization for six mice (25 μl for each mouse). Above mentioned formulations were further analyzed by transmission electron microscopy for measuring size distribution. CoVpep-AuNP conjugation was confirmed by measuring the shift in absorbance wavelength by UV-vis spectrophotometer. % conjugation of CoVpep on the surface of AuNPs were determined by BCA assay.

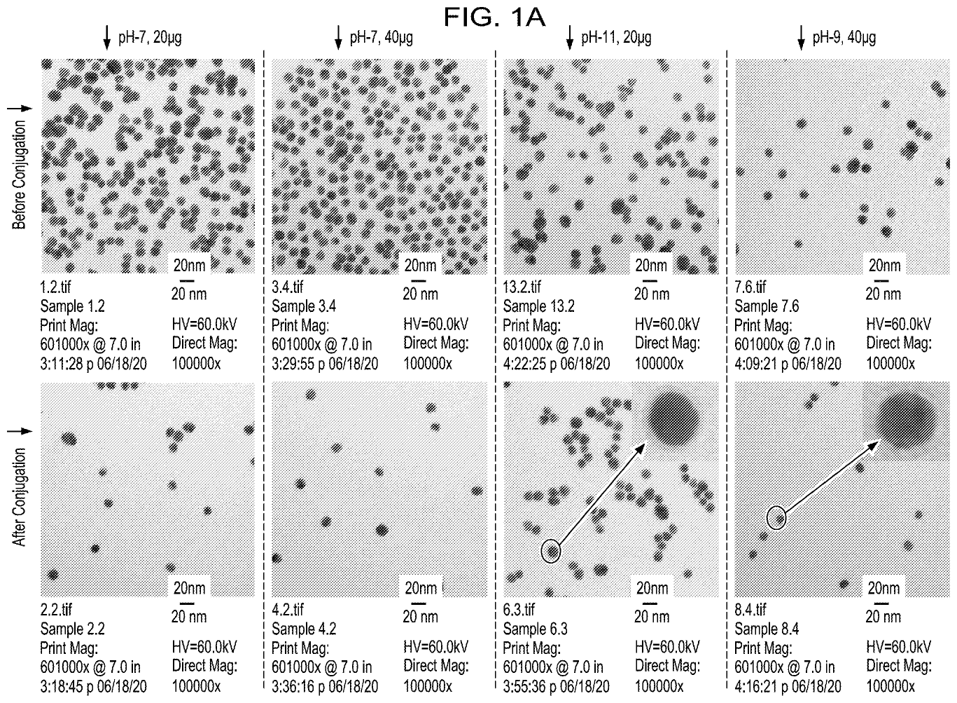

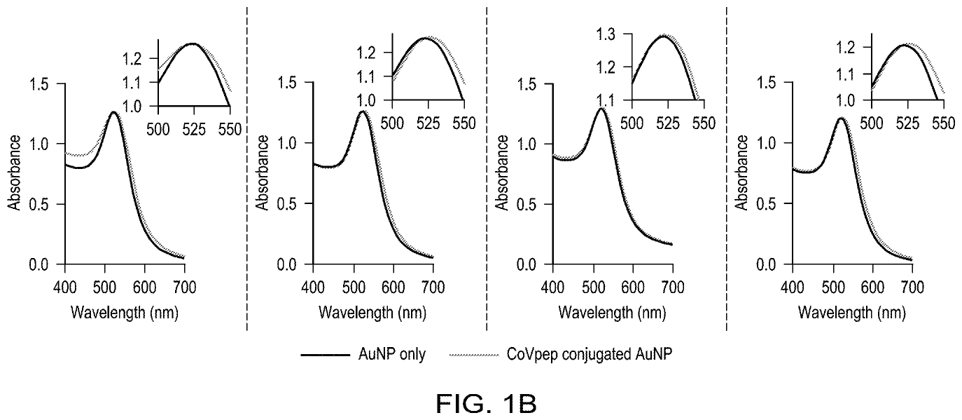

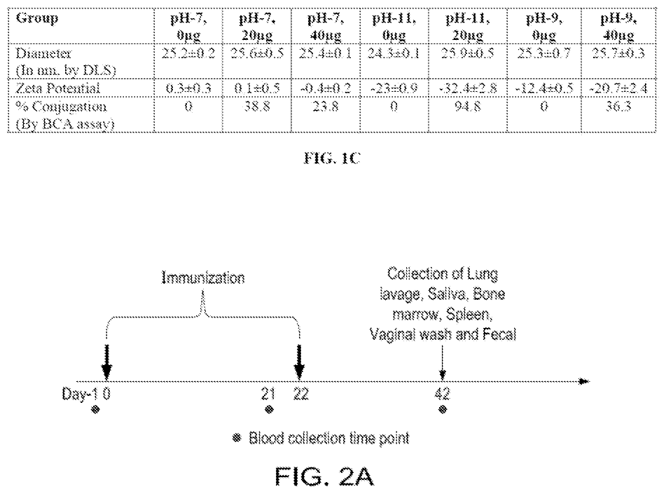

FIGS. 1A to 1C shows the results from Antigen 1 being conjugated to gold nanoparticles at different pH conditions. FIG. 1A shows TEM images of gold nanoparticles (AuNP) before (upper images) and after the conjugation (lower images) with the fusion protein of Antigen 1 (CoVpep), under different conditions and amounts. FIG. 1B shows UV-Vis spectra of the AuNP before and after conjugation with CoVpep. FIG. 1C shows the diameter, zeta potential, and percent conjugation before and after conjugation of the AuNP and CoVpep.

Mice were vaccinated twice, once on day 0 and then on day 21. Analysis of antibody responses and the ability of the antibodies to bind to different coronaviruses was evaluated. It was found that a good antibody response was observed when Antigen 1 was attached to gold nanoparticles and CpG was used as an adjuvant. The serum from vaccinated mice was able to bind to not only SARS-COV-2 (COVID-19 causative agent) but also to other coronaviruses.

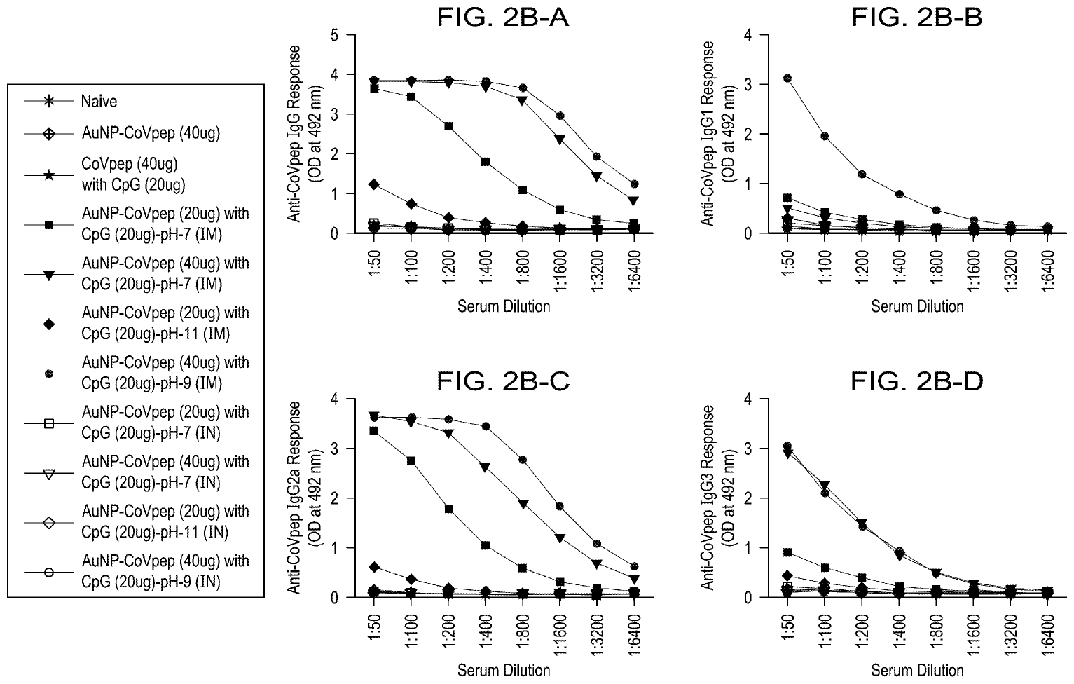

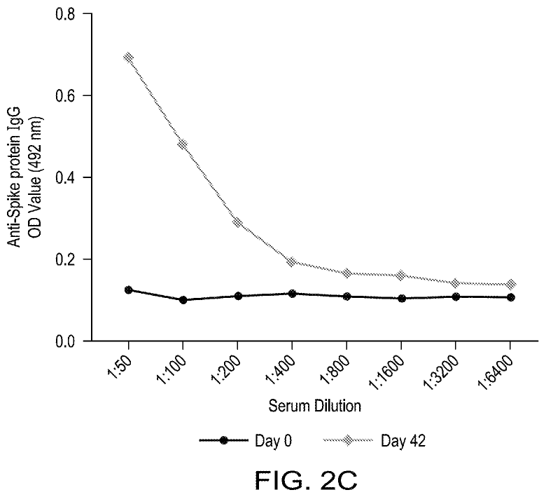

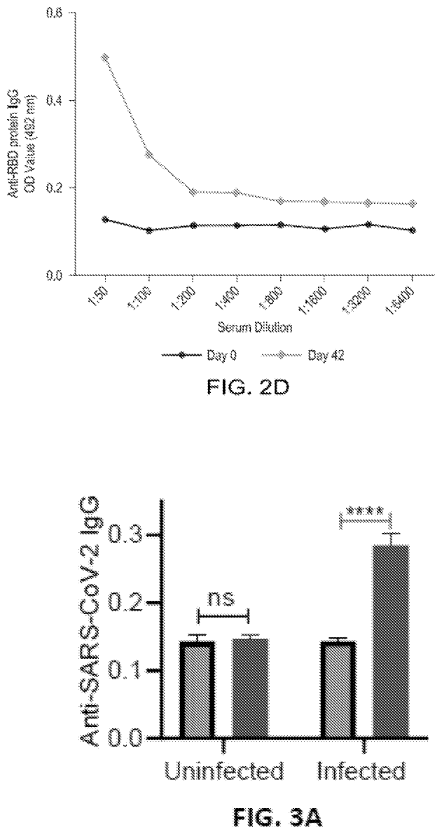

FIGS. 2A to 2D show the immunization schedule and results from the immunization of mice with the Antigen 1 of the present invention. FIG. 2A shows the immunization schedule and sample collection points. FIG. 2B includes four graphs (2A-A to 2A-D) that show the results from Anti-CoVpep serum antibody titration curve at day-42. FIG. 2C is a graph that shows anti-Spike protein serum antibody titration curve at day-42. FIG. 2D is a graph that shows anti-RBD serum antibody titration curve at day-42.

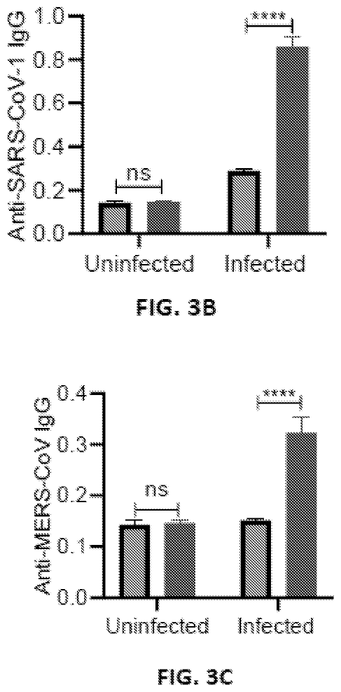

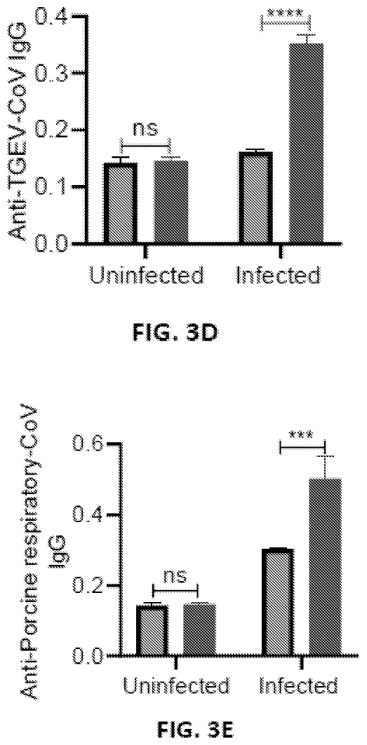

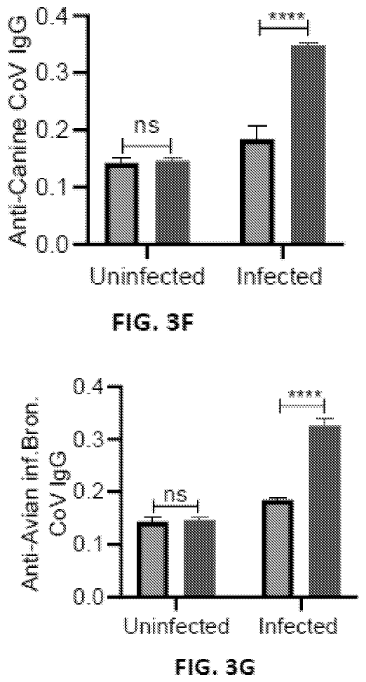

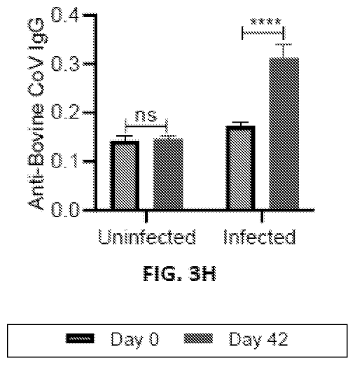

FIGS. 3A to 3H show the cross-reactivity of serum antibodies against different strains of CoV at Day 0 and Day 42. FIG. 3A anti-SARS-CoV-2 IgG. FIG. 3B anti-SARS-CoV-1 IgG. FIG. 3C anti-MERS-CoV IgG. FIG. 3D anti-TEGV-CoV IgG. FIG. 3E anti-porcine respiratory-CoV IgG. FIG. 3F anti-canine-CoV IgG. FIG. 3G anti-avian infectious bronchitis-CoV IgG. FIG. 3H anti-Bovine-CoV-2 IgG.

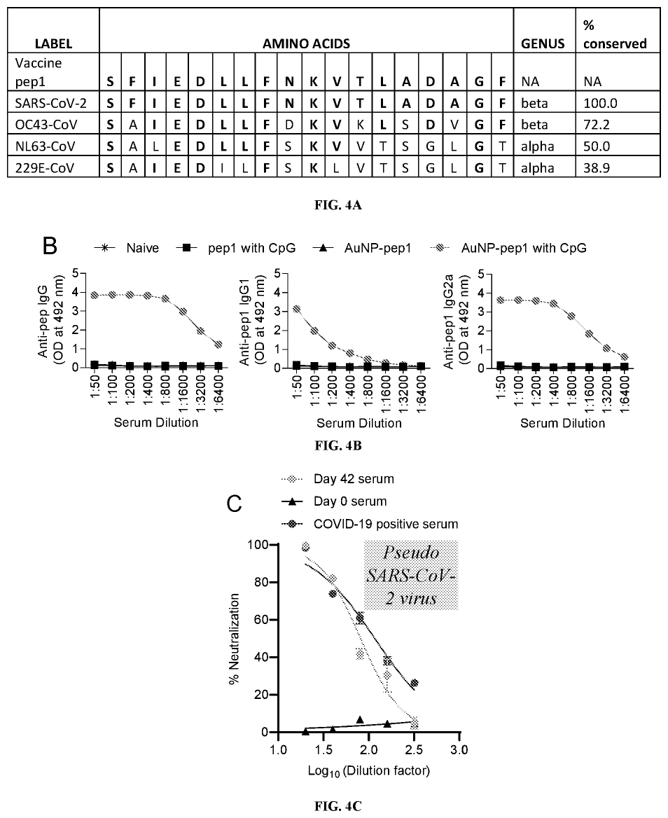

To study the immune response from the conserved pep1 peptide, the inventors vaccinated balb/c mice (n=5, 6-8 week old) twice (21 day apart) through an intramuscular injection. Pep1 was attached to gold nanoparticles and, CpG (CpG 1826: 5′-TCCATGACGTTCCTGACGTT-3′)(SEQ ID NO:18), a class B oligodeoxynucleotide was added as an adjuvant. CpG is a TLR9 agonist and stimulates a strong Th1-biased response towards antigens. Each dose contained ˜60 μg AuNP, 40 μg pep1, and 20 μg CpG. As controls, groups of mice received either pep1 with CpG or AuNPs with pep1 attached (but no CpG was added). A naïve group was also included. The degree of similarity of pep1 to SARS-CoV-2 virus and few other CoVs is shown in FIG. 4A. The vaccinated mice receiving the AuNP-pep1 with CpG formulation generated a significantly higher anti-pep1 IgG, IgG1, and IgG2 response as compared to the groups of mice receiving either ‘pep1 with CpG’ or ‘AuNP-pep1 (without CpG)’ (FIG. 4B). Consistent with the activity of CpG as a Th1-biased adjuvant the inventors observed a higher IgG2a (surrogate marker of Th1 response) response as compared to IgG1 (a surrogate marker of Th2 response) antibody response.

FIGS. 4A to 4E show the antibody response, cross reactivity and virus neutralization from conserved spike pep1 peptide. Balb/c mice, 6-8 week old were vaccinated intramuscularly (IM) on day 0 and 21 with 40 μg pep1 conjugated on ˜60 μg AuNPs and 20 μg CpG. Day 42 serum was analyzed. (FIG. 4A) Sequence alignment of pep1 with different CoVs from alpha and beta genera, SEQ ID NOS:16, 16, 19, 20, and 21. (FIG. 4B) IgG, IgG1 and IgG2a antibody response towards pep1. (FIG. 4C) Neutralization of a pseudotyped reporter lentivirus containing SARS-CoV-2 spike protein on the surface and a GFP reporter gene. Mouse sera was heat inactivated at 56° C. for 30 min and added at two-fold dilutions (starting 1:20 dilution) to a fixed amount of reporter virus. After 1 h incubation the incubation mixture was added to confluent HEK293T cells expressing human ACE2 receptor in 96 well plates. After 72 h of culture the cells were run through FACS to determine percent of GFP expressing cells, which was then converted to % neutralization and reported. At low serum dilution (more antibodies) higher % neutralization is observed. (FIG. 4D) Live virus neutralization assay. Method from Poh et. al [71] was followed. Briefly, mouse sera was heat inactivated at 56° C. for 30 min and added at two-fold dilutions (starting 1:20 dilution) to a fixed amount (100 TCID50) of different strains of CoVs (human CoV-OC43, human CoV-NL63 and human CoV 229E). After 1 h incubation the incubation mixture was added to cell monolayers in 96 well plates (HCT-8 cells for human CoV-OC43, LLCMK2 cells for human CoV-NL63 and MRC-5 cells for human CoV-229E strains). After 7 days of culture incubation, the cell viability of each well was determined using Viral ToxGlo Assay (Promega, #G8941) to determine relative luminescence unit (RLU) using microplate reader (Biotek synergy H1). This RLU was then normalized and converted to % neutralization and reported. At low serum dilution (more antibodies) higher % neutralization is observed. (FIG. 4E) Infected-cell-based ELISA. Method from Conzelmann et. al [72] was followed. Briefly, cross reactivity of antibody response from pep1-based vaccine was assessed by measuring IgG binding to spike protein expressed in different live CoV infected cell lines. Confluent monolayers of HCT-8 cells, LLCMK2 cells, and MRC-5 cells were infected with human CoV-OC43, human CoV-NL63 and human CoV-229E strains, respectively, for 72 h, 72 h and 48 h incubation time at 34° C. and 5% CO₂. Next, supernatant from each well was removed and cells were washed 3 times with PBS and fixed with 4% paraformaldehyde for 30 min. After fixation, the cells were washed and permeabilized with 0.1% Triton-X-100 for 5 min. After permeabilization, the cells were blocked with 3% BSA solution for 2 h at RT. After washing with PBST, the day 0 and day 42 immunized serum were incubated at 1:50 dilution for 1.5 h at room temperature (RT). The wells were then washed with PBST and incubated with HRP conjugated anti mouse IgG at 1:4000 dilution for 1.5 h at RT. After washing with PBST, OPD substrate was added for 15 min, and OD was measured at 492 nm.

To test whether the serum antibodies from pep1 vaccinated mice can neutralize SARS-CoV-2 the inventors used a BSL2 lentivirus pseudovirus that contained SARS-CoV-2 spike protein on its surface to infect cells and a GFP reporter gene for readout of the assay (Integral Molecular, PA). HEK293T cells that express hACE2 on their surface were cultured in 96 well plates. Mouse serum was diluted two-fold starting at 1:20 dilution and incubated for 1 h with pseudo reporter virus (same amount was used for all samples). As a control, a sample of the pseudovirus without any antibody was used. These mixtures were then added to the 96 well plates containing HEK293T cells. After 72 h of culture, the cells were detached and run through FACS. As a positive control, serum from a human who had recovered from COVID-19 was used, while day 0 (before vaccination) mouse serum was used as a negative control. Just the cells without any addition of virus or serum were also used as a control. To benchmark no (zero) neutralization, the pseudovirus without antibodies was used. The FACS readout was converted to % neutralization based on the fraction of total cells that were positive for GFP[71]. The resulting neutralization data is shown in FIG. 4C. Antibodies from pep1 vaccinated mice clearly demonstrated neutralization of the SARS-CoV-2 pseudovirus similar to convalescent plasma sample.

The inventors reasoned that the conserved peptides identified herein would be able to provide cross-neutralization against different CoV strains as long as there is a good match between them. To test this, the inventors attempted to neutralize three other human CoV strains: OC43 (beta), NL63 (alpha) and 229E (alpha). From FIG. 4A it can be seen that the pep1 (a beta strain consensus sequence) has a good match (72%) with OC43 (also a beta strain) while with NL63 and 229E, which are both alpha strains, the pep1 amino acid sequence homology is just 50% and 38.9%, respectively. FIG. 4D shows the neutralization titer for serum obtained from pep1-vaccinated mice (AuNP-pep1+CpG group). Indeed, the serum showed good neutralization against wild type OC43 CoV, but as expected the neutralization titer dropped for wild type NL63 and 229E in proportion with reduction in their respective homology between pep1 and the corresponding sequence on spike protein of NL63 and 229E. These result provides strong rigor and supports that linear conserved epitopes can generate a cross protective immune response.

Bioimmunoinformatics for identifying CoV spike conserved epitopes. To make a broadly protective CoV vaccine, it is important to include antigens that are conserved across different CoV genera. To identify the conserved epitopes of the spike protein and nucleoprotein, the inventors obtained reference amino acid sequences of the seven known human CoVs (1 NL63, 1 229E, 2 MERS, 2 SARS, 1 SARS-COV-2, 1 HKU1, and 1 OC43) as FASTA files from NCBI Virus database[77]. Using the bioinformatics toolbox of MATLAB and custom code also written in MATLAB, the inventors performed ‘multiple alignment’ for the spike protein and nucleoprotein. When alpha and beta strains were analyzed together, poor homology was observed. However, high homology was observed when the analysis was separately performed for alpha and beta genera. The consensus sequences for alpha strains show a better homology since only two huma alpha viruses are known as compared to 5 different human beta CoVs. This analysis shows that while alpha CoVs differ from beta CoVs, yet within each genera there is good homology and these sequences can be used to develop a broadly protective CoV vaccine. NOTE: Different human CoVs use different receptors (aminopeptidase N (APN)^{[78]}, sialic acid[79], (DPP4)[80], and angiotensin-converting enzyme 2 (ACE2)[81, 82]) to bind target cells for infection. Since this interaction occurs through spike receptor binding domain (RBD), RBD is inherently different in different CoVs. Therefore, when we aligned the spike proteins, no conserved region in RBD was identified. This is good for vaccine design because current mutations are being observed in RBD domain (like India (delta), South Africa, UK, Brazil/Japan mutant). By not using the RBD domain as an immunogen, the vaccine can stay unaffected by these mutations. However, we did find that the fusion domain had good homology and consensus epitopes were found.

Tables 2A and 2B show the bioimmunoinformatics of spike protein and nucleoprotein of alpha and beta human CoV reference strains. Human alpha Coronavirus CoV consensus sequences spike protein: SEQ ID NOS:22-28. Human alpha Coronavirus CoV consensus sequences nucleoprotein: SEQ ID NOS:29-30. Spike protein and nucleoprotein amino acid sequences for human alpha and beta CoV reference strains were downloaded from NCBI virus database[77]. These sequences were multiple aligned using custom code written in MATLAB. Regions of spike protein and nucleoprotein that were most conserved in the different strains were identified. The amino acid numbering shown is for NL63 CoV for alpha genera (spike: ‘YP_003767’ and nucleoprotein: ‘YP_003771’), and for SARS-CoV-2 Wuhan for beta genera (spike: ‘YP_009724390.1’ and nucleoprotein: ‘YP_009724397’). Human beta Coronavirus CoV consensus sequences spike protein: SEQ ID NOS:31-35. Human beta Coronavirus CoV consensus sequences nucleoprotein: SEQ ID NOS:36-39. The % values provide the range of consensus the epitopes have with different alpha and beta strains. IEDB database B & T Cell Receptor epitopes: Bold: common region for potential B and T cell epitope; Non-bold: only T-cell epitope.

TABLE 2A
Human Alpha CoV Consensus Sequences

Human alpha CoV consensus sequences
Spike Protein SEQ ID NOS: 22-28
T A N L S I P S N W T T S V Q V E Y L Q I T S T P I V V D C aa 760-789
L L K Q Y T S A C K T I E D A L R aa 803-819
S A I E D I L F S K I V T S G L G T fusion domain aa 871-888 aa 896-936
C T K G L S I A D L A C A Q Y Y N G I M V L P G V A D A E R M A M Y T G S L I G G
Q A R L N Y V A L Q T D V L Q E N Q K I L A A S F N K A aa 955-982
A L N K I Q D V V N Q Q G N A L N H L T S Q L R aa 1010-1033
S I Q A I Y D R L D S I Q A D Q Q V D R L I T G R L A A L N aa 1042-1071
Nucleoprotein SEQ ID NOS: 29-30
V I P R N L V P I G K aa 36-46
R V D L P P K I H F Y Y L G T G P H K D A K F R Q R aa 69-94

TABLE 2A
Human Beta CoV Consensus Sequences

Human beta CoV consensus sequences
Spike Protein SEQ ID NOS: 31-35
S F I E D L L F D K V T L A D A G F aa 816-833 fusion domain
R D L I C A Q K F N G L K V L P P L L aa 847-865
Y R F N G I G V T Q N V L Y E N Q K L I A N Q F N Q A L aa 904-931
K I Q D V V N Q N A Q A L N T L aa 947-962
Q I D R L I N G R L Q S L N A Y V T Q Q L aa 992-1012
Nucleoprotein SEQ ID NOS: 36-39
S W F T G L T Q H G K aa 51-61
K Q L S P R W Y F Y Y L G T G P E A aa 102-119
G T T L P K G F Y V E G S aa 164-176
K P R Q K R T A T K Q Y N V T Q A F G R R G P aa 257-279

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±0.1, 0.5, 1, 2, 3, 4, 5, 6, 7, 10, 12 or 15%, or as understood to be within a normal tolerance in the art, for example, within 2 standard deviations of the mean. Unless otherwise clear from the context, all numerical values provided herein are modified by the term about.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. King, A. M. Q., et al., Changes to taxonomy and the International Code of Virus Classification and Nomenclature ratified by the International Committee on Taxonomy of Viruses (2018). Arch Virol, 2018. 163(9): p. 2601-2631.
2. Li, W., et al., Bats are natural reservoirs of SARS-like coronaviruses. Science, 2005. 310(5748): p. 676-679.
3. Kusanagi, K.-i., et al., Isolation and serial propagation of porcine epidemic diarrhea virus in cell cultures and partial characterization of the isolate. Journal of Veterinary Medical Science, 1992. 54(2): p. 313-318.
4. Poon, L. L. M., et al., Identification of a novel coronavirus in bats. Journal of virology, 2005. 79(4): p. 2001-2009.
5. Kndelovi, M., et al., Detection of Murine Herpesvirus 68 (MHV-68) in Dermacentor reticulatus ticks. Microbial ecology, 2015. 70(3): p. 785-794.
6. Cui, J., F. Li, and Z.-L. Shi, Origin and evolution of pathogenic coronaviruses. Nature reviews Microbiology, 2019. 17(3): p. 181-192.
7. Drexler, J. F., V. M. Corman, and C. Drosten, Ecology, evolution and classification of bat coronaviruses in the aftermath of SARS. Antiviral research, 2014. 101: p. 45-56.
8. Pedersen, N. C., An update on feline infectious peritonitis: virology and immunopathogenesis. The veterinary journal, 2014. 201(2): p. 123-132.
9. Ma, Y., et al., Origin, evolution, and virulence of porcine deltacoronaviruses in the United States. MBio, 2015. 6(2): p. e00064-15.
10. Woo, P. C. Y., et al., Discovery of a novel bottlenose dolphin coronavirus reveals a distinct species of marine mammal coronavirus in Gammacoronavirus. Journal of virology, 2014. 88(2): p. 1318-1331.
11. Woo, P. C. Y., et al., Comparative analysis of complete genome sequences of three avian coronaviruses reveals a novel group 3c coronavirus. Journal of virology, 2009. 83(2): p. 908-917.
12. Woo, P. C. Y., et al., Discovery of seven novel Mammalian and avian coronaviruses in the genus deltacoronavirus supports bat coronaviruses as the gene source of alphacoronavirus and betacoronavirus and avian coronaviruses as the gene source of gammacoronavirus and deltacoronavirus. Journal of virology, 2012. 86(7): p. 3995-4008.
13. Chiu, S. S., et al., Human coronavirus NL63 infection and other coronavirus infections in children hospitalized with acute respiratory disease in Hong Kong, China. Clinical infectious diseases, 2005. 40(12): p. 1721-1729.
14. Hamre, D. and J. J. Procknow, A new virus isolated from the human respiratory tract. Proceedings of the Society for Experimental Biology and Medicine, 1966. 121(1): p. 190-193.
15. Ksiazek, T. G., et al., A novel coronavirus associated with severe acute respiratory syndrome. New Englandjournal of medicine, 2003. 348(20): p. 1953-1966.
16. Vabret, A., et al., An outbreak of coronavirus OC43 respiratory infection in Normandy, France. Clinical infectious diseases, 2003. 36(8): p. 985-989.
17. Drosten, C., et al., Identification of a novel coronavirus in patients with severe acute respiratory syndrome. New England journal of medicine, 2003. 348(20): p. 1967-1976.
18. Zaki, A. M., et al., Isolation of a novel coronavirus from a man with pneumonia in Saudi Arabia. New England Journal of Medicine, 2012. 367(19): p. 1814-1820.
19. Du, L., et al., Vaccines for the prevention against the threat of MERS-CoV. Expert review of vaccines, 2016. 15(9): p. 1123-1134.
20. Woo, P. C. Y., et al., Characterization and complete genome sequence of a novel coronavirus, coronavirus HKU1, from patients with pneumonia. Journal of virology, 2005. 79(2): p. 884-895.
21. Zhang, N., et al., Recent advances in the detection of respiratory virus infection in humans. Journal of Medical Virology, 2020. 92(4): p. 408-417.
22. Zhu, N., et al., A novel coronavirus from patients with pneumonia in China, 2019. New England Journal of Medicine, 2020.
23. Johns Hopkins Coronavirus Resource Center. https://coronavirus.jhu.edu/map.html; last accessed Jul. 14, 2020.
24. Liu, S.-L. and L. Saif, Emerging viruses without borders: The Wuhan coronavirus. 2020, Multidisciplinary Digital Publishing Institute.
25. Fehr, A. R. and S. Perlman, Coronaviruses: an overview of their replication and pathogenesis, in Coronaviruses. 2015, Springer. p. 1-23.
26. Snijder, E. J., E. Decroly, and J. Ziebuhr, The nonstructural proteins directing coronavirus RNA synthesis and processing, in Advances in virus research. 2016, Elsevier. p. 59-126.
27. Menachery, V. D., et al., MERS-CoV accessory ORFs play key role for infection and pathogenesis. MBio, 2017. 8(4): p. e00665-17.
28. Lu, L., et al., Structure-based discovery of Middle East respiratory syndrome coronavirus fusion inhibitor. Nature communications, 2014. 5(1): p. 1-12.
29. Li, F., et al., Structure of SARS coronavirus spike receptor-binding domain complexed with receptor. Science, 2005. 309(5742): p. 1864-1868.
30. Hulswit, R. J. G., et al., Human coronaviruses OC43 and HKU1 bind to 9-O-acetylated sialic acids via a conserved receptor-binding site in spike protein domain A. Proceedings of the National Academy of Sciences, 2019. 116(7): p. 2681-2690.
31. Lu, G., et al., Molecular basis of binding between novel human coronavirus MERS-CoV and its receptor CD26. Nature, 2013. 500(7461): p. 227-231.
32. Li, F., Receptor recognition mechanisms of coronaviruses: a decade of structural studies. Journal of virology, 2015. 89(4): p. 1954-1964.
33. Li, F., Structure, function, and evolution of coronavirus spike proteins. Annual review of virology, 2016. 3: p. 237-261.
34. Yeager, C. L., et al., Human aminopeptidase N is a receptor for human coronavirus 229E. Nature, 1992. 357(6377): p. 420-422.
35. Vlasak, R., et al., Human and bovine coronaviruses recognize sialic acid-containing receptors similar to those of influenza C viruses. Proceedings of the National Academy of Sciences, 1988. 85(12): p. 4526-4529.
36. Zhou, P., et al., A pneumonia outbreak associated with a new coronavirus of probable bat origin. Nature, 2020: p. 1-4.
37. Li, W., et al., Angiotensin-converting enzyme 2 is a functional receptor for the SARS coronavirus. Nature, 2003. 426(6965): p. 450-454.
38. Raj, V. S., et al., Dipeptidyl peptidase 4 is a functional receptor for the emerging human coronavirus-EMC. Nature, 2013. 495(7440): p. 251-254.
https://www.frontiersin.org/articles/10.3389/fimmu.2018.02224/full
https://www.nature.com/articles/s41565-020-0737-y
https://www.sciencedirect.com/science/article/pii/S181808761400035X
https://pubs.rsc.org/en/content/articlelanding/2020/nr/c9nr08958f#!divAbstract
https://www.nature.com/articles/s41565-020-0737-y
https://www.ncbi.nlm.nih.gov/pmc/articles/PMC22232/
https://www.futuremedicine.com/doi/10.2217/nnm-2018-0147
https://pubmed.ncbi.nlm.nih.gov/23829488/
https://pubmed.ncbi.nlm.nih.gov/25842219/

1. An immunogenic composition comprising:
a nanoparticle conjugated to one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response.

2. The formulation of claim 1, further comprising at least one of:
an adjuvant selected from at least one of: monophosphoryl lipid A, synthetic lipid A, lipid A mimetics or analogs, aluminum salts, cytokines, saponins, muramyl dipeptide, N-glycolyl dipeptide, polyIC, polyCpG, lipopolysaccharide, polyphosphazenes, emulsions, virosomes, virus like particles, bacteria, algae, yeast, cochleates, poly(lactide-co-glycolides) microparticles, poloxamer particles, microparticles, toll-like receptor agonists, helper T cell agonists, T cell stimulating peptides added to the composition, T cell stimulating peptides conjugated or fused to the one or more antigenic peptides or fusion polypeptides, water in oil emulsion, oil in water emulsion, resiquimod, inulin, algammulin, lipid particles, or liposomes;
one or more spherical particles or any other regular or irregular shape with a mean of its largest dimension being below 1000 micrometers, below 100 micrometers, and more preferably below 10 micrometers, and below 1 micrometer, and below 0.5 micrometer, and the deviation of the particles being less than 75% of the mean, less than 50% of the mean, less than 25% of the mean or less than 15% of the mean;
a buffer selected from the group consisting of phosphate buffer, citrate buffer, phosphate citrate buffer, borate buffer, tris(hydroxymethyl)aminomethane (Tris) containing buffer, succinate buffer, and buffers containing glycine or histidine as one of the buffering agents; or
the composition is formulated for a mucosal, intranasal, intramuscular, intravenous, intrapulmonary, enteric, oral, subcutaneous, intradermal, subdermal, or transdermal route of administration.

3. (canceled)

4. The composition of claim 1, wherein the nanoparticle comprises the antigenic peptides or polypeptides, wherein the antigenic peptides or polypeptides are crosslinked, precipitating the antigenic peptides or polypeptides, aggregating the antigenic peptides or polypeptides, the particle is made of a different material such as metals or their oxides or their salts (gold, silver, iron oxide, aluminum hydroxide, aluminum phosphate), synthetic polymers (poly(lactide-co-glycolide), polycapralactone, polyanhydrides), inorganic molecules (silica), metal particles, zoonotic viruses, human viruses, bacterial viruses, plant viruses, bacteria, bacterial or fungal spores, yeast, liposomes, lipids, or other proteins and peptides that self-assemble, DNA/RNA molecules that self-assemble, pollen shells, carbohydrates, sugars, virus-like particles, or any combination of the aforementioned, a mixture of heterogenous particles made from one or more materials, the materials combined with a coating or a polymer layer on the metal particles, or by coating gold over silica particles, or combinations thereof.

5. The composition of claim 1, wherein the antigenic peptides or polypeptides are at least one of:
mixed with particles, the antigenic peptides or polypeptides are chemically attached to a particle surface, or the antigenic peptides or polypeptides are entrapped in the particle core, or a combination thereof, wherein the antigenic peptides or polypeptides are in a free form and in an attached form;
are made synthetically, recombinantly, in a prokaryotic expression system or a eukaryotic expression system;
are expressed in a prokaryotic expression system or a eukaryotic expression system;
are separated by a linker; or
are selected from at least one of SEQ ID NOS: 1 to 16, 22 to 39, any combination and concatemers thereof.

6. (canceled)

7. (canceled)

8. (canceled)

9. (canceled)

10. (canceled)

11. The composition of claim 1, wherein the composition at least one of:
elicits two or more immune responses selected from: immunoglobulin production, Th1 protective immunity, Th2 protective immunity, or any combination thereof;
is in a liquid or a lyophilized form; or
is contained within pre-filled syringes, microneedle patch, needle-free patch, and/or inhalation or nasal sprays.

12. (canceled)

13. (canceled)

14. (canceled)

15. The composition of claim 1, wherein the virus is selected from a rhinovirus, coronavirus, paramyxoviridae, Orthomyxoviridae, adenovirus, parainfluenza virus, metapneumovirus, respiratory syncytial virus or influenza virus.

16. A method of eliciting protective immunity to a viral infection in a mammal or avian comprising administering to the mammal or avian a vaccine comprising a nanoparticle conjugated to one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response.

17. The method of claim 16, further comprising at least one of:
an adjuvant selected from at least one of: monophosphoryl lipid A, synthetic lipid A, lipid A mimetics or analogs, aluminum salts, cytokines, saponins, muramyl dipeptide, N-glycolyl dipeptide, polyIC, polyCpG, lipopolysaccharide, polyphosphazenes, emulsions, virosomes, virus like particles, bacteria, algae, yeast, cochleates, poly(lactide-co-glycolides) microparticles, poloxamer particles, microparticles, toll-like receptor agonists, helper T cell agonists, T cell stimulating peptides added to the composition, T cell stimulating peptides conjugated or fused to the one or more antigenic peptides or fusion polypeptides, water in oil emulsion, oil in water emulsion, resiquimod, inulin, algammulin, lipid particles, or liposomes;
one or more spherical particles or any other regular or irregular shape with a mean of its largest dimension being below 1000 micrometers, below 100 micrometers, and more preferably below 10 micrometers, and below 1 micrometer, and below 0.5 micrometer, and the deviation of the particles being less than 75% of the mean, less than 50% of the mean, less than 25% of the mean or less than 15% of the mean;
a buffer selected from the group consisting of phosphate buffer, citrate buffer, phosphate citrate buffer, borate buffer, tris(hydroxymethyl)aminomethane (Tris) containing buffer, succinate buffer, and buffers containing glycine or histidine as one of the buffering agents; or
the composition is formulated for a mucosal, intranasal, intramuscular, intravenous, intrapulmonary, enteric, oral, subcutaneous, intradermal, subdermal, or transdermal route of administration.

18. The method of claim 16, wherein the nanoparticle is a gold or silver nanoparticle, or a metal coated nanoparticle.

19. (canceled)

20. The method of claim 16, wherein the nanoparticle comprises the antigenic peptides or polypeptides, wherein the antigenic peptides or polypeptides are crosslinked, precipitating the antigenic peptides or polypeptides, aggregating the antigenic peptides or polypeptides, the particle is made of a different material such as metals or their oxides or their salts (gold, silver, iron oxide, aluminum hydroxide, aluminum phosphate), synthetic polymers (poly(lactide-co-glycolide), polycapralactone, polyanhydrides), inorganic molecules (silica), metal particles, zoonotic viruses, human viruses, bacterial viruses, plant viruses, bacteria, bacterial or fungal spores, yeast, liposomes, lipids, or other proteins and peptides that self-assemble, DNA/RNA molecules that self-assemble, pollen shells, carbohydrates, sugars, virus-like particles, or any combination of the aforementioned, a mixture of heterogenous particles made from one or more materials, the materials combined with a coating or a polymer layer on the metal particles, or by coating gold over silica particles, or combinations thereof.

21. The method of claim 16, wherein the antigenic peptides or polypeptides are at least one of:
mixed with particles, and the antigenic peptides or polypeptides are chemically attached to a particle surface, or the antigenic peptides or polypeptides are entrapped in the particle core, or a combination thereof, wherein the antigenic peptides or polypeptides are in a free form and in an attached form;
are made synthetically, recombinantly, in a prokaryotic expression system or a eukaryotic expression system;
are expressed in a prokaryotic expression system or a eukaryotic expression system;
are separated by a linker; or
are selected from at least one of SEQ ID NOS: 1 to 16, 22 to 39, any combination and concatemers thereof.

22. (canceled)

23. (canceled)

24. (canceled)

25. (canceled)

26. The method of claim 16, wherein the composition at least one of:
elicits two or more immune responses selected from: immunoglobulin production, Th1 protective immunity, Th2 protective immunity, or any combination thereof;
is in a liquid or a lyophilized form; or
is contained within pre-filled syringes, microneedle patch, needle-free patch, and/or inhalation or nasal sprays.

27. (canceled)

28. (canceled)

29. (canceled)

30. The method of claim 16, wherein the vaccine is in a dose amount of from about 1 microgram to about 1 gram.

31. The method of claim 16, wherein the virus is selected from a rhinovirus, coronavirus, paramyxoviridae, Orthomyxoviridae, adenovirus, parainfluenza virus, metapneumovirus, respiratory syncytial virus or influenza virus.

32. An immunogenic formulation comprising:
an antigenic peptide or fusion polypeptide comprising: (Aₙ₁Bₙ₂Cₙ₃Dₙ₄Eₙ₅Fₙ₆Gₙ₇Hₙ₈Iₙ₉Jₙ₁₀Kₙ₁₁)ₙ₁₂ wherein:

| Name | Protein | SEQ ID NO: |
|---|---|---|
| A | Spike_1 | 1 |
| B | Spike_2 | 2 |
| C | Spike_3 | 3 |
| D | Spike_4 | 4 |
| E | Spike_5 | 5 |
| F | Matrix_1 | 6 |
| G | NP_1 | 7 |
| H | RDRP_1 | 8 |
| I | RDRP_2 | 9 |
| J | RDRP_3 | 10 |
| K | RDRP_4 | 11 |

wherein n1, n2, n3, n4, n5, n6, n7, n8, n9, n10, and n11 can be any digit greater than or equal to zero but all are not simultaneously equal to zero, and the order of A, B, C, D, E, F, G, H, I, J, and K can be in any permutation and combination and wherein n12 is greater than zero, wherein the peptides are optionally separated by a linker.

33. The formulation of claim 32, further comprising at least one of:
one or more spherical particles or any other regular or irregular shape with a mean of its largest dimension being below 1000 micrometers, below 100 micrometers, and more preferably below 10 micrometers, and below 1 micrometer, and below 0.5 micrometer, and the deviation of the particles being less than 75% of the mean, less than 50% of the mean, less than 25% of the mean or less than 15% of the mean;
a buffer selected from the group consisting of phosphate buffer, citrate buffer, phosphate citrate buffer, borate buffer, tris(hydroxymethyl)aminomethane (Tris) containing buffer, succinate buffer, and buffers containing glycine or histidine as one of the buffering agents;
the composition is formulated for a mucosal, intranasal, intramuscular, intravenous, intrapulmonary, enteric, oral, subcutaneous, intradermal, subdermal, or transdermal route of administration;
one or more atoms or one or more molecules are placed at the amino terminus, the carboxy terminus, between one or more amino acids, between one or more one or more peptides, or any combination thereon, wherein when two or more atoms or two or more molecules are included, they can be the same or different atoms or molecules, the atom is selected from any of the known elements of the periodic table, or the atom is selected from gold or silver.

34. The formulation of claim 32, wherein the nanoparticle comprises the antigenic peptides or polypeptides, wherein the antigenic peptides or polypeptides are crosslinked, precipitating the antigenic peptides or polypeptides, aggregating the antigenic peptides or polypeptides, the particle is made of a different material such as metals or their oxides or their salts (gold, silver, iron oxide, aluminum hydroxide, aluminum phosphate), synthetic polymers (poly(lactide-co-glycolide), polycapralactone, polyanhydrides), inorganic molecules (silica), metal particles, zoonotic viruses, human viruses, bacterial viruses, plant viruses, bacteria, bacterial or fungal spores, yeast, liposomes, lipids, or other proteins and peptides that self-assemble, DNA/RNA molecules that self-assemble, pollen shells, carbohydrates, sugars, virus-like particles, or any combination of the aforementioned, a mixture of heterogenous particles made from one or more materials, the materials combined with a coating or a polymer layer on the metal particles, or by coating gold over silica particles, or combinations thereof.

35. The formulation of claim 32, wherein the antigenic peptides or polypeptides are at least one of:
mixed with particles, the antigenic peptides or polypeptides are chemically attached to a particle surface, or the antigenic peptides or polypeptides are entrapped in the particle core, or a combination thereof, wherein the antigenic peptides or polypeptides are in a free form and in an attached form;
are made synthetically, recombinantly, in a prokaryotic expression system or a eukaryotic expression system;
are expressed in a prokaryotic expression system or a eukaryotic expression system;
are separated by a linker;
are selected from at least one of SEQ ID NOS: 1 to 16, 22 to 39, any combination and concatemers thereof; or
comprises 2, 3, 4, 5, 6, 7, 8, 9, 10, 11, 12, 13, 14, 15, 16 or more antigenic peptides or polypeptides.

36. (canceled)

37. (canceled)

38. (canceled)

39. (canceled)

40. (canceled)

41. The formulation of claim 32, wherein the one or more molecules is one or more fat, one or more lipid, one or more carbohydrate, one or more natural or synthetic amino acid, one or more peptide, one or more protein, one or more nucleotide, one or more polymer synthetic or natural, or any combination thereof.

42. The formulation of claim 32, further comprising an adjuvant selected from at least one of: monophosphoryl lipid A, synthetic lipid A, lipid A mimetics or analogs, aluminum salts, cytokines, saponins, muramyl dipeptide, N-glycolyl dipeptide, polyIC, polyCpG, lipopolysaccharide, polyphosphazenes, emulsions, virosomes, virus like particles, bacteria, algae, yeast, cochleates, poly(lactide-co-glycolides) microparticles, poloxamer particles, microparticles, toll-like receptor agonists, helper T cell agonists, T cell stimulating peptides added to the composition, T cell stimulating peptides conjugated or fused to the one or more antigenic peptides or fusion polypeptides, water in oil emulsion, oil in water emulsion, resiquimod, inulin, algammulin, lipid particles, or liposomes.

43. (canceled)

44. (canceled)

45. (canceled)

46. The formulation of claim 32, wherein the composition at least one of:
elicits two or more immune responses selected from: immunoglobulin production, Th1 protective immunity, Th2 protective immunity, or any combination thereof;
is in a liquid or a lyophilized form; or
is contained within pre-filled syringes, microneedle patch, needle-free patch, and/or inhalation or nasal sprays.

47. (canceled)

48. (canceled)

49. (canceled)

50. (canceled)

51. A formulation comprising the molecule (A*ₙ₁B*ₙ₂C*ₙ₃D*ₙ₄E*ₙ₅F*ₙ₆G*ₙ₇H*ₙ₈*ₙ₉J*ₙ₁₀K*ₙ₁₁)ₙ₁₂, wherein A*, B*, C*, D*, E*, F*, G*, H*, I*, J*, K* are each a portion of contiguous amino acids selected from:

| Name | Protein | SEQ ID NO: |
|---|---|---|
| A | Spike_1 | 1 |
| B | Spike_2 | 2 |
| C | Spike_3 | 3 |
| D | Spike_4 | 4 |
| E | Spike_5 | 5 |
| F | Matrix_1 | 6 |
| G | NP_1 | 7 |
| H | RDRP_1 | 8 |
| I | RDRP_2 | 9 |
| J | RDRP_3 | 10 |
| K | RDRP_4 | 11 |

wherein n1, n2, n3, n4, n5, n6, n7, n8, n9, n10, and n11 can be any digit greater than or equal to zero but all are not simultaneously equal to zero, and the order of A*, B*, C*, D*, E*, F*, G*, H*, I*, J*, K* can be in any permutation and combination and n12 is greater than zero, wherein the peptides are optionally separated by a linker.

52. A method of making an immunogenic composition comprising:
selecting one or more antigenic peptides or fusion polypeptides from more than one strain of virus, wherein the one or more antigenic peptides or fusion polypeptides elicit at least one of a humoral, Th1, Th2 or CTL immune response; and
synthesizing or expressing the one or more antigenic peptides or fusion polypeptides for the immunogenic composition.

53. A nucleic acid that encodes one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response.

54. The nucleic acid of claim 53, wherein the nucleic acid is formulated into a composition is formulated into a vaccine for a mucosal, intranasal, intramuscular, intravenous, intrapulmonary, enteric, oral, subcutaneous, intradermal, subdermal, or transdermal route of administration.

55. The nucleic acid of claim 53, wherein the one or more antigenic peptides or fusion polypeptides expressed by the nucleic acid are at least one of:
mixed with particles, and the antigenic peptides or polypeptides are chemically attached to a particle surface, or the antigenic peptides or polypeptides are entrapped in the particle core, or a combination thereof, wherein the antigenic peptides or polypeptides are in a free form and in an attached form;
are made synthetically, recombinantly, in a prokaryotic expression system or a eukaryotic expression system;
are expressed in a prokaryotic expression system or a eukaryotic expression system;
are separated by a linker;
are selected from at least one of SEQ ID NOS: 1 to 16, 22 to 39, any combination and concatemers thereof; or
expressed in a prokaryotic expression system or a eukaryotic expression system.

56. (canceled)

57. The nucleic acid of claim 53, wherein the one or more antigenic peptides or fusion polypeptides are selected from at least one of SEQ ID NOS: 1 to 16, 22 to 39 any combination and concatemers thereof.

58. The nucleic acid of claim 53, wherein the nucleic acid is formulated into a composition elicits two or more immune responses selected from: immunoglobulin production, Th1 protective immunity, Th2 protective immunity, or any combination thereof.

59. A host cell comprising a nucleic acid that encodes one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response.

60. A nucleic acid expression vector that encodes one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response.

61. A vaccine comprising a nucleic acid that encodes one or more antigenic peptides or fusion polypeptides from more than one strain of virus, wherein the one or more antigenic peptides or fusion polypeptides elicit at least one of a humoral, T helper cell-1 (Th1), T helper cell-2 (Th2) or cytotoxic T cell (CTL) immune response, wherein the vaccine is an RNA or a DNA vaccine.

62. A method of immunizing a subject comprising injecting the subject with an amount of a nucleic acid that encodes one or more antigenic peptides or fusion polypeptides from more than one strain of coronavirus, wherein the one or more antigenic peptides or fusion polypeptides elicit at least one of a humoral, T helper cell-1 (Th1), T helper cell-2 (Th2) or cytotoxic T cell (CTL) immune response, sufficient to trigger an immune response to the one or more antigenic peptides or fusion polypeptides, wherein the vaccine is an RNA or a DNA vaccine.


SEQUENCE LISTING (42 sequences)

SEQ ID NO: 1 - protein, 41 aa, Coronavirus sp.
  SITE (1)..(41): Spike_1
RSFIEDLLFNKVTLADAGFMKQYGDCLGDIAARDLICAQKF

SEQ ID NO: 2 - protein, 22 aa, Coronavirus sp.
  SITE (1)..(22): Spike_2
YRFNGIGVTQNVLYENQKLIAN

SEQ ID NO: 3 - protein, 14 aa, Coronavirus sp.
  SITE (1)..(14): Spike_3
KLQDVVNQNAQALN

SEQ ID NO: 4 - protein, 37 aa, Coronavirus sp.
  SITE (1)..(37): Spike_4
QLSSNFGAISSVLNDILSRLDKVEAEVQIDRLITGRL

SEQ ID NO: 5 - protein, 10 aa, Coronavirus sp.
  SITE (1)..(10): Spike_5
YIKWPWYIWL

SEQ ID NO: 6 - protein, 27 aa, Coronavirus sp.
  SITE (1)..(27): Matrix_1
MWLSYFIASFRLFARTRSMWSFNPETN

SEQ ID NO: 7 - protein, 12 aa, Coronavirus sp.
  SITE (1)..(12): NP_1
PRWYFYYLGTGP

SEQ ID NO: 8 - protein, 11 aa, Coronavirus sp.
  SITE (1)..(11): RDRP_1
VGVLTLDNQDL

SEQ ID NO: 9 - protein, 12 aa, Coronavirus sp.
  SITE (1)..(12): RDRP_2
LMGWDYPKCDRA

SEQ ID NO: 10 - protein, 11 aa, Coronavirus sp.
  SITE (1)..(11): RDRP_3
KHFSMMILSDD

SEQ ID NO: 11 - protein, 22 aa, Coronavirus sp.
  SITE (1)..(22): RDRP_4
TQMNLKYAISAKNRARTVAGVS

SEQ ID NO: 12 - protein, 400 aa, Artificial Sequence
  Synthetic
KKKKVGVLTLDNQDLVGVLTLDNQDLVGVLTLDNQDLVGVLTLDNQDLVGVLTLDNQDLKKKKLMGWDYPKCDRALMGWDYPKCDRALMGWDYPKCDRALMGWDYPKCDRALMGWDYPKCDRAKKKKHFSMMILSDDKHFSMMILSDDKHFSMMILSDDKHFSMMILSDDKHFSMMILSDDKKKKKTQMNLKYAISAKNRARTVAGVSTQMNLKYAISAKNRARTVAGVSTQMNLKYAISAKNRARTVAGVSTQMNLKYAISAKNRARTVAGVSTQMNLKYAISAKNRARTVAGVSKKKKSFIEDLLFNKVSFIEDLLFNKVSFIEDLLFNKVSFIEDLLFNKVSFIEDLLFNKVKKKKFYYLGTGPFYYLGTGPFYYLGTGPFYYLGTGPFYYLGTGPC

SEQ ID NO: 13 - protein, 1096 aa, Artificial Sequence
  Synthetic
RSFIEDLLFNKVTLADAGFMKQYGDCLGDIAARDLICAQKFRSFIEDLLFNKVTLADAGFMKQYGDCLGDIAARDLICAQKFRSFIEDLLFNKVTLADAGFMKQYGDCLGDIAARDLICAQKFRSFIEDLLFNKVTLADAGFMKQYGDCLGDIAARDLICAQKFRSFIEDLLFNKVTLADAGFMKQYGDCLGDIAARDLICAQKFYRFNGIGVTQNVLYENQKLIANYRFNGIGVTQNVLYENQKLIANYRFNGIGVTQNVLYENQKLIANYRFNGIGVTQNVLYENQKLIANYRFNGIGVTQNVLYENQKLIANKLQDVVNQNAQALNKLQDVVNQNAQALNKLQDVVNQNAQALNKLQDVVNQNAQALNKLQDVVNQNAQALNQLSSNFGAISSVLNDILSRLDKVEAEVQIDRLITGRLQLSSNFGAISSVLNDILSRLDKVEAEVQIDRLITGRLQLSSNFGAISSVLNDILSRLDKVEAEVQIDRLITGRLQLSSNFGAISSVLNDILSRLDKVEAEVQIDRLITGRLQLSSNFGAISSVLNDILSRLDKVEAEVQIDRLITGRLYIKWPWYIWLYIKWPWYIWLYIKWPWYIWLYIKWPWYIWLYIKWPWYIWLMWLSYFIASFRLFARTRSMWSFNPETNMWLSYFIASFRLFARTRSMWSFNPETNMWLSYFIASFRLFARTRSMWSFNPETNMWLSYFIASFRLFARTRSMWSFNPETNMWLSYFIASFRLFARTRSMWSFNPETNVGVLTLDNQDLVGVLTLDNQDLVGVLTLDNQDLVGVLTLDNQDLVGVLTLDNQDLLMGWDYPKCDRALMGWDYPKCDRALMGWDYPKCDRALMGWDYPKCDRALMGWDYPKCDRAKHFSMMILSDDKHFSMMILSDDKHFSMMILSDDKHFSMMILSDDKHFSMMILSDDTQMNLKYAISAKNRARTVAGVSTQMNLKYAISAKNRARTVAGVSTQMNLKYAISAKNRARTVAGVSTQMNLKYAISAKNRARTVAGVSTQMNLKYAISAKNRARTVAGVSPRWYFYYLGTGPPRWYFYYLGTGPPRWYFYYLGTGPPRWYFYYLGTGPPRWYFYYLGTGPC

SEQ ID NO: 14 - protein, 1366 aa, Artificial Sequence
  Synthetic
RSFIEDLLFNKVTLADAGFMKQYGDCLGDIAARDLICAQKFGGDGGRSFIEDLLFNKVTLADAGFMKQYGDCLGDIAARDLICAQKFGGDGGRSFIEDLLFNKVTLADAGFMKQYGDCLGDIAARDLICAQKFGGDGGRSFIEDLLFNKVTLADAGFMKQYGDCLGDIAARDLICAQKFGGDGGRSFIEDLLFNKVTLADAGFMKQYGDCLGDIAARDLICAQKFGGDGGYRFNGIGVTQNVLYENQKLIANGGDGGYRFNGIGVTQNVLYENQKLIANGGDGGYRFNGIGVTQNVLYENQKLIANGGDGGYRFNGIGVTQNVLYENQKLIANGGDGGYRFNGIGVTQNVLYENQKLIANGGDGGKLQDVVNQNAQALNGGDGGKLQDVVNQNAQALNGGDGGKLQDVVNQNAQALNGGDGGKLQDVVNQNAQALNGGDGGKLQDVVNQNAQALNGGDGGQLSSNFGAISSVLNDILSRLDKVEAEVQIDRLITGRLGGDGGQLSSNFGAISSVLNDILSRLDKVEAEVQIDRLITGRLGGDGGQLSSNFGAISSVLNDILSRLDKVEAEVQIDRLITGRLGGDGGQLSSNFGAISSVLNDILSRLDKVEAEVQIDRLITGRLGGDGGQLSSNFGAISSVLNDILSRLDKVEAEVQIDRLITGRLGGDGGYIKWPWYIWLGGDGGYIKWPWYIWLGGDGGYIKWPWYIWLGGDGGYIKWPWYIWLGGDGGYIKWPWYIWLGGDGGMWLSYFIASFRLFARTRSMWSFNPETNGGDGGMWLSYFIASFRLFARTRSMWSFNPETNGGDGGMWLSYFIASFRLFARTRSMWSFNPETNGGDGGMWLSYFIASFRLFARTRSMWSFNPETNGGDGGMWLSYFIASFRLFARTRSMWSFNPETNGGDGGVGVLTLDNQDLGGDGGVGVLTLDNQDLGGDGGVGVLTLDNQDLGGDGGVGVLTLDNQDLGGDGGVGVLTLDNQDLGGDGGLMGWDYPKCDRAGGDGGLMGWDYPKCDRAGGDGGLMGWDYPKCDRAGGDGGLMGWDYPKCDRAGGDGGLMGWDYPKCDRAGGDGGKHFSMMILSDDGGDGGKHFSMMILSDDGGDGGKHFSMMILSDDGGDGGKHFSMMILSDDGGDGGKHFSMMILSDDGGDGGTQMNLKYAISAKNRARTVAGVSGGDGGTQMNLKYAISAKNRARTVAGVSGGDGGTQMNLKYAISAKNRARTVAGVSGGDGGTQMNLKYAISAKNRARTVAGVSGGDGGTQMNLKYAISAKNRARTVAGVSGGDGGPRWYFYYLGTGPGGDGGPRWYFYYLGTGPGGDGGPRWYFYYLGTGPGGDGGPRWYFYYLGTGPGGDGGPRWYFYYLGTGPC

SEQ ID NO: 15 - protein, 1366 aa, Artificial Sequence
  Synthetic
RSFIEDLLFNKVTLADAGFMKQYGDCLGDIAARDLICAQKFGPGPGRSFIEDLLFNKVTLADAGFMKQYGDCLGDIAARDLICAQKFGPGPGRSFIEDLLFNKVTLADAGFMKQYGDCLGDIAARDLICAQKFGPGPGRSFIEDLLFNKVTLADAGFMKQYGDCLGDIAARDLICAQKFGPGPGRSFIEDLLFNKVTLADAGFMKQYGDCLGDIAARDLICAQKFGPGPGYRFNGIGVTQNVLYENQKLIANGPGPGYRFNGIGVTQNVLYENQKLIANGPGPGYRFNGIGVTQNVLYENQKLIANGPGPGYRFNGIGVTQNVLYENQKLIANGPGPGYRFNGIGVTQNVLYENQKLIANGPGPGKLQDVVNQNAQALNGPGPGKLQDVVNQNAQALNGPGPGKLQDVVNQNAQALNGPGPGKLQDVVNQNAQALNGPGPGKLQDVVNQNAQALNGPGPGQLSSNFGAISSVLNDILSRLDKVEAEVQIDRLITGRLGPGPGQLSSNFGAISSVLNDILSRLDKVEAEVQIDRLITGRLGPGPGQLSSNFGAISSVLNDILSRLDKVEAEVQIDRLITGRLGPGPGQLSSNFGAISSVLNDILSRLDKVEAEVQIDRLITGRLGPGPGQLSSNFGAISSVLNDILSRLDKVEAEVQIDRLITGRLGPGPGYIKWPWYIWLGPGPGYIKWPWYIWLGPGPGYIKWPWYIWLGPGPGYIKWPWYIWLGPGPGYIKWPWYIWLGPGPGMWLSYFIASFRLFARTRSMWSFNPETNGPGPGMWLSYFIASFRLFARTRSMWSFNPETNGPGPGMWLSYFIASFRLFARTRSMWSFNPETNGPGPGMWLSYFIASFRLFARTRSMWSFNPETNGPGPGMWLSYFIASFRLFARTRSMWSFNPETNGPGPGVGVLTLDNQDLGPGPGVGVLTLDNQDLGPGPGVGVLTLDNQDLGPGPGVGVLTLDNQDLGPGPGVGVLTLDNQDLGPGPGLMGWDYPKCDRAGPGPGLMGWDYPKCDRAGPGPGLMGWDYPKCDRAGPGPGLMGWDYPKCDRAGPGPGLMGWDYPKCDRAGPGPGKHFSMMILSDDGPGPGKHFSMMILSDDGPGPGKHFSMMILSDDGPGPGKHFSMMILSDDGPGPGKHFSMMILSDDGPGPGTQMNLKYAISAKNRARTVAGVSGPGPGTQMNLKYAISAKNRARTVAGVSGPGPGTQMNLKYAISAKNRARTVAGVSGPGPGTQMNLKYAISAKNRARTVAGVSGPGPGTQMNLKYAISAKNRARTVAGVSGPGPGPRWYFYYLGTGPGPGPGPRWYFYYLGTGPGPGPGPRWYFYYLGTGPGPGPGPRWYFYYLGTGPGPGPGPRWYFYYLGTGPC

SEQ ID NO: 16 - protein, 23 aa, Artificial Sequence
  Synthetic
SFIEDLLFNKVTLADAGFKKKKC

SEQ ID NO: 17 - protein, 18 aa, Coronavirus sp.
  SITE (1)..(18): portion of spike 1
SFIEDLLFNKVTLADAGF

SEQ ID NO: 18 - DNA, 20 bp, Artificial Sequence
  Synthetic CpG
tccatgacgttcctgacgtt

SEQ ID NO: 19 - protein, 18 aa, Coronavirus sp.
SAIEDLLFDKVKLSDVGF

SEQ ID NO: 20 - protein, 18 aa, Coronavirus sp.
SALEDLLFSKVVTSGLGT

SEQ ID NO: 21 - protein, 18 aa, Coronavirus sp.
SAIEDILFSKLVTSGLGT

SEQ ID NO: 22 - protein, 30 aa, Coronavirus sp.
  SITE (1)..(30): Human alpha CoV consensus sequence
TANLSIPSNWTTSVQVEYLQITSTPIVVDC

SEQ ID NO: 23 - protein, 17 aa, Coronavirus sp.
  SITE (1)..(17): Human alpha CoV consensus sequence
LLKQYTSACKTIEDALR

SEQ ID NO: 24 - protein, 18 aa, Coronavirus sp.
  SITE (1)..(18): Human alpha CoV consensus sequence
SAIEDILFSKIVTSGLGT

SEQ ID NO: 25 - protein, 42 aa, Coronavirus sp.
  SITE (1)..(42): Human alpha CoV consensus sequence
CTKGLSIADLACAQYYNGIMVLPGVADAERMBAMYTGSLIGG

SEQ ID NO: 26 - protein, 28 aa, Coronavirus sp.
  SITE (1)..(28): Human alpha CoV consensus sequence
QARLNYVALQTDVLQEBQKILAASFNKA

SEQ ID NO: 27 - protein, 24 aa, Coronavirus sp.
  SITE (1)..(24): Human alpha CoV consensus sequence
ALNKIQDVVNQQGNALNHLTSQLR

SEQ ID NO: 28 - protein, 30 aa, Coronavirus sp.
  SITE (1)..(30): Human alpha CoV consensus sequence
SIQAIYDRLDSIQADQQVDRLITGRLAALN

SEQ ID NO: 29 - protein, 11 aa, Coronavirus sp.
  SITE (1)..(11): Human alpha CoV consensus sequence
VIPRNLVPIGK

SEQ ID NO: 30 - protein, 27 aa, Coronavirus sp.
  SITE (1)..(27): Human alpha CoV consensus sequence
RRVDLPPKIHFYYLGTGPHKDAKFRQR

SEQ ID NO: 31 - protein, 18 aa, Coronavirus sp.
  SITE (1)..(18): Human beta CoV consensus sequence
SFIEDLLFDKVTLADAGF

SEQ ID NO: 32 - protein, 19 aa, Coronavirus sp.
  SITE (1)..(19): Human beta CoV consensus sequence
RDLICAQKFNGLKVLPPLL

SEQ ID NO: 33 - protein, 28 aa, Coronavirus sp.
  SITE (1)..(28): Human beta CoV consensus sequence
YRFNGIGVTQNVLYENQKLIANQFNQAL

SEQ ID NO: 34 - protein, 16 aa, Coronavirus sp.
  SITE (1)..(16): Human beta CoV consensus sequence
KIQDVVNQNAQALNTL

SEQ ID NO: 35 - protein, 21 aa, Coronavirus sp.
  SITE (1)..(21): Human beta CoV consensus sequence
QIDRLINGRLQSLNAYVTQQL

SEQ ID NO: 36 - protein, 11 aa, Coronavirus sp.
  SITE (1)..(11): Human beta CoV consensus sequence
SWFTGLTQHGK

SEQ ID NO: 37 - protein, 18 aa, Coronavirus sp.
  SITE (1)..(18): Human beta CoV consensus sequence
KQLSPRWYFYYLGTGPEA

SEQ ID NO: 38 - protein, 13 aa, Coronavirus sp.
  SITE (1)..(13): Human beta CoV consensus sequence
GTTLPKGFYVEGS

SEQ ID NO: 39 - protein, 23 aa, Coronavirus sp.
  SITE (1)..(23): Human beta CoV consensus sequence
KPRQKRTATKQYNVTQAFGRRGP

SEQ ID NO: 40 - protein, 4 aa, Artificial Sequence
  Synthetic linker
KKKK

SEQ ID NO: 41 - protein, 5 aa, Artificial Sequence
  Synthetic linker
GGDGG

SEQ ID NO: 42 - protein, 5 aa, Artificial Sequence
  Synthetic linker
GPGPG